(19) United States
(12) Patent Application Publication
(10) Pub. No.: US 2026/0305975 A1
(43) Pub. Date: Oct. 8, 2026

(54) SYSTEMS AND METHODS FOR PERSONALIZED BREATH BASED GLYCEMIC MONITORING AND PROJECTED A1C TREND ESTIMATION

(71) Applicant: PreEvnt, LLC, Oxnard, CA (US)

(72) Inventors: Clinton Wilcox, Nevada City, CA (US); Fredrick T. Brooks, III, Ventura, CA (US)

(21) Appl. No.: 19/635,284

(22) Filed: Mar. 31, 2026

Related U.S. Application Data
(60) Provisional application No. 63/783,138, filed on Apr. 3, 2025.
(60) Provisional application No. 63/782,491, filed on Apr. 2, 2025.

(51) Int. Cl. A61B 5/083; A61B 5/00; A61B 10/00; G01N 33/497

(52) U.S. Cl. CPC A61B 5/083; A61B 5/6822; A61B 5/7221; A61B 5/7246; A61B 5/7275; A61B 5/742; A61B 10/00; G01N 33/4975; A61B 2010/0087; A61B 2560/0223; A61B 2560/0247

(57) ABSTRACT

A non-invasive glycemic monitoring system uses a breath-sensing device to acquire repeated breath sessions from a user and to generate volatile organic compound information together with sample-quality and environmental information. A computing system receives a lab-derived A1C baseline for the user and uses the baseline with the breath-derived information to initialize and update a personalized model. Accepted breath sessions are stored in a rolling history and used to generate a projected A1C trend and a daily progress score or zone output representative of the user’s glycemic trajectory. In some embodiments, humidity, temperature, and sample-quality information are used to normalize breath-session data, and optional external inputs including finger-stick data, continuous glucose monitor data, or other external data are fused with the breath-derived information to refine personalization, anomaly handling, reporting, and confirmatory prompts.

### CROSS-REFERENCE TO RELATED APPLICATIONS

This application claims the benefit under 35 U.S.C. § 119(e) of U.S. Provisional Patent Application No. 63/782,491, filed April 2, 2025, and U.S. Provisional Patent Application No. 63/783,138, filed April 3, 2025, the entire disclosures of which are hereby incorporated herein by reference.

### BACKGROUND OF THE DISCLOSURE

### Technical Field of the Disclosure

The present disclosure relates generally to non-invasive metabolic monitoring and, more particularly, to systems and methods for personalized glycemic monitoring, projected A1C trend estimation, and related data fusion using breath-derived volatile organic compound information.

### Background and Description of the Related Art

The present disclosure relates generally to non-invasive metabolic monitoring and, more particularly, to systems and methods that use breath-derived volatile organic compound (VOC) information to support personalized glycemic monitoring and longer-term glycemic trend estimation.

Glycemic health is commonly evaluated using laboratory A1C testing, finger-stick glucose testing, and, in some cases, continuous glucose monitoring. Laboratory A1C testing can provide a useful indication of longer-term glycemic exposure, but it is ordinarily performed only intermittently. As a result, such testing may provide limited day-to-day feedback regarding how meals, exercise, stress, sleep, medication use, or other behaviors are affecting a person’s glycemic trajectory between lab visits.

Finger-stick testing and other conventional monitoring techniques may provide more immediate information, but can be episodic, inconvenient, invasive, or otherwise undesirable for some users. Continuous glucose monitoring systems can provide richer short-term data, but typically require a sensor worn on or inserted into the body and may present comfort, compliance, calibration, maintenance, and cost considerations. Accordingly, many users, particularly users with pre-diabetes, type 2 diabetes, early metabolic dysfunction, or users attempting to reduce glucose excursions, may benefit from a non-invasive alternative that can be used repeatedly in ordinary daily life.

Users may also benefit from a monitoring framework that does more than simply present isolated readings. For many users, a simple daily output indicating whether recent behavior appears to be improving or worsening longer-term glycemic status may be more actionable than occasional laboratory values alone. A personalized progress indicator, zone, trend, or similar output can help users understand the cumulative effect of repeated high-glucose events and may encourage earlier lifestyle modification or confirmatory clinical follow-up.

Exhaled breath contains VOCs associated with metabolic processes, and breath-based sensing has been investigated as a non-invasive source of physiological information. However, converting breath measurements into useful glycemic information presents practical and technical challenges. Breath measurements may be affected by sample quality, breathing conditions, ambient conditions, humidity, temperature, device configuration, and inter-user physiological variation. In addition, a generalized model may not adequately reflect the baseline characteristics or ongoing changes of a particular user.

There remains a need for systems and methods capable of acquiring repeat breath measurements, evaluating sample validity and environmental conditions, deriving glucose-correlated metrics from the breath measurements, and personalizing interpretation of such metrics over time. There is further need for approaches that can make use of a known laboratory benchmark, such as a recent A1C value, as an anchor for subsequent non-invasive monitoring rather than attempting to rely solely on an out-of-the-box population estimate.

There is also a need for user-friendly architectures that can maintain rolling histories, generate daily progress or zone-based outputs, project longer-term trends, identify anomalous sessions, prompt confirmatory testing when appropriate, and generate reports suitable for user review or sharing with clinicians. Optional integration with other data sources, such as finger-stick values, continuous glucose monitoring data, or external data feeds, may further improve personalization and cross-checking.

In addition, flexible breath-sensing platforms may be useful in contexts extending beyond glucose-oriented monitoring, including broader VOC analytics and condition-screening workflows. Accordingly, there remains a need for improved non-invasive monitoring systems that combine repeatable breath-sensing, personalization, longitudinal analysis, and practical reporting in a form suitable for consumer, wellness, screening, and clinical-support environments.

### SUMMARY OF THE INVENTION

The present disclosure provides systems and methods for non-invasive monitoring of glycemic status using volatile organic compound (VOC) information obtained from a user’s breath. In certain embodiments, a wearable or handheld breath-sensing device acquires one or more breath samples from a user and processes the resulting sensor data to derive a glucose-correlated breath metric. The system may then use that metric to generate one or more user-facing outputs, such as a glycemic zone, a progress score, a projected A1C-related indicator, an alert, or a report.

In certain embodiments, the system is personalized using a laboratory-derived A1C value obtained for the user at or near onboarding. Rather than relying solely on a generalized out-of-the-box estimate, the system may use the lab A1C value as an anchor for initializing or refining a user-specific model. The model may thereafter be updated over time based on repeated breath sessions, thereby allowing the system to provide a daily or other recurring indication of whether the user’s glycemic trajectory appears to be improving, remaining stable, or worsening.

In certain embodiments, the system includes a breath-sensing device, a mobile application, and optionally a remote data store or other computing infrastructure. The breath-sensing device may include a housing, a breath inlet region, one or more filters or barriers, a VOC sensor, and one or more additional sensors configured to detect environmental or quality-related conditions, such as humidity, temperature, pressure, or other contextual information. The system may use such additional information to evaluate sample quality, normalize measurements, reject anomalous sessions, improve confidence, or otherwise increase the reliability of derived outputs.

In certain embodiments, the system stores a rolling history of breath-derived results and uses the rolling history to generate a projected A1C trend, a daily progress score, a risk category, or a zone-based output. The output may be presented in a simplified manner for consumer use, such as by colored zones or ranked categories, while also supporting more detailed views such as dashboards, trend histories, projected A1C displays, notes, alerts, and exportable reports. In some embodiments, the system may prompt a confirmatory test, such as a laboratory test or other clinical follow-up, when one or more thresholds, anomaly conditions, or concerning trends are identified.

In certain embodiments, the system further supports optional integration of additional glucose-related inputs. For example, the system may ingest one or more laboratory values, continuous glucose monitor data, finger-stick data, or externally provided data streams through manual entry, wireless transfer, application programming interfaces, or other interfaces. A personalization or data-fusion engine may use such inputs together with breath-derived information to refine model performance, cross-check anomalies, generate projected A1C-related outputs, and produce reports for users, clinicians, or other authorized recipients.

In certain embodiments, the disclosure also preserves broader VOC-analytics architectures in which a breath sensor device performs VOC feature extraction and provides information to a database, classifier, model, retraining workflow, enterprise platform, or related diagnostic infrastructure. Such broader architectures may be used in combination with the glycemic-monitoring embodiments described herein or in other VOC-analysis contexts.

The foregoing summary is provided to introduce selected concepts in simplified form and is not intended to identify every feature of the claimed subject matter or to limit the scope of the claimed subject matter. Additional features, combinations, and embodiments will be apparent from the drawings, the detailed description, and the claims.

These and other features, embodiments, and advantages will be apparent from the following description and drawings.

### BRIEF DESCRIPTION OF THE DRAWINGS

Elements in the figures have not necessarily been drawn to scale in order to enhance their clarity and improve understanding of these various elements and embodiments of the invention. Furthermore, elements that are known to be common and well understood to those in the industry are not depicted in order to provide a clear view of the various embodiments of the invention.

FIG. 1 is a schematic system overview of an example breath-based glycemic monitoring system including a breath-sensing device, a mobile application, a remote data store, a personalization engine, and example outputs.

FIG. 2A is a front perspective view of an example pendant device.

FIG. 2B is a perspective view of an example pendant device with a lanyard or clasp embodiment.

FIG. 3 is an exploded view of example internal components of the pendant device.

FIG. 4 is a flow diagram of an example onboarding and personalization process including obtaining a lab A1C baseline, acquiring one or more breath samples, evaluating sample quality and environmental data, deriving a glucose-correlated breath metric, initializing a personalized model, storing baseline and rolling history, and producing an initial zone or progress output.

FIG. 5 is a flow diagram of an example daily monitoring process including acquiring a new breath session, normalizing the session using temperature, humidity, and sample quality, updating rolling history, projecting an A1C trend, computing a daily progress score, performing a threshold or anomaly check, displaying zone, report, and alert output, and optionally prompting a confirmatory test.

FIG. 6 is a schematic view of example user-interface and reporting views including a dashboard view, a zone indicator, a projected A1C display, a history or trend view, a notes control, and a report or export view.

FIG. 7 is a schematic view of an example data-fusion architecture integrating breath-derived input with a lab A1C anchor and optional external inputs to generate a projected A1C output, a daily score or zone output, an anomaly cross-check, and a report or export output.

FIG. 8 is a schematic view of a broader VOC analytics architecture including a breath sensor device, VOC feature extraction, a VOC pattern database, a classifier or model, a condition likelihood output, model update or retraining, and an enterprise or cloud platform.

### DETAILED DESCRIPTION OF THE DRAWINGS

In the following discussion that addresses a number of embodiments and applications of the present invention, reference is made to the accompanying drawings that form a part hereof, and in which is shown by way of illustration specific embodiments in which the invention may be practiced. It is to be understood that other embodiments may be utilized, and changes may be made without departing from the scope of the present invention.

Various inventive features are described below that can each be used independently of one another or in combination with other features. However, any single inventive feature may not address any of the problems discussed above or only address one of the problems discussed above. Further, one or more of the problems discussed above may not be fully addressed by any of the features described below.

As used herein, the singular forms "a", "an" and "the" include plural referents unless the context clearly dictates otherwise. All embodiments of any aspect of the invention can be used in combination, unless the context clearly dictates otherwise.

Unless the context clearly requires otherwise, throughout the description and the claims, the words ‘comprise’, ‘comprising’, and the like are to be construed in an inclusive sense as opposed to an exclusive or exhaustive sense; that is to say, in the sense of “including, but not limited to”. Words using the singular or plural number also include the plural and singular number, respectively. Additionally, the words “herein,” “wherein”, “whereas” “above,” and “below” and words of similar import, when used in this application, shall refer to this application as a whole and not to any particular portions of the application.

The description of embodiments of the disclosure is not intended to be exhaustive or to limit the disclosure to the precise form disclosed. While the specific embodiments of, and examples for, the disclosure are described herein for illustrative purposes, various equivalent modifications are possible within the scope of the disclosure, as those skilled in the relevant art will recognize.

### Overall System and Operating Environment

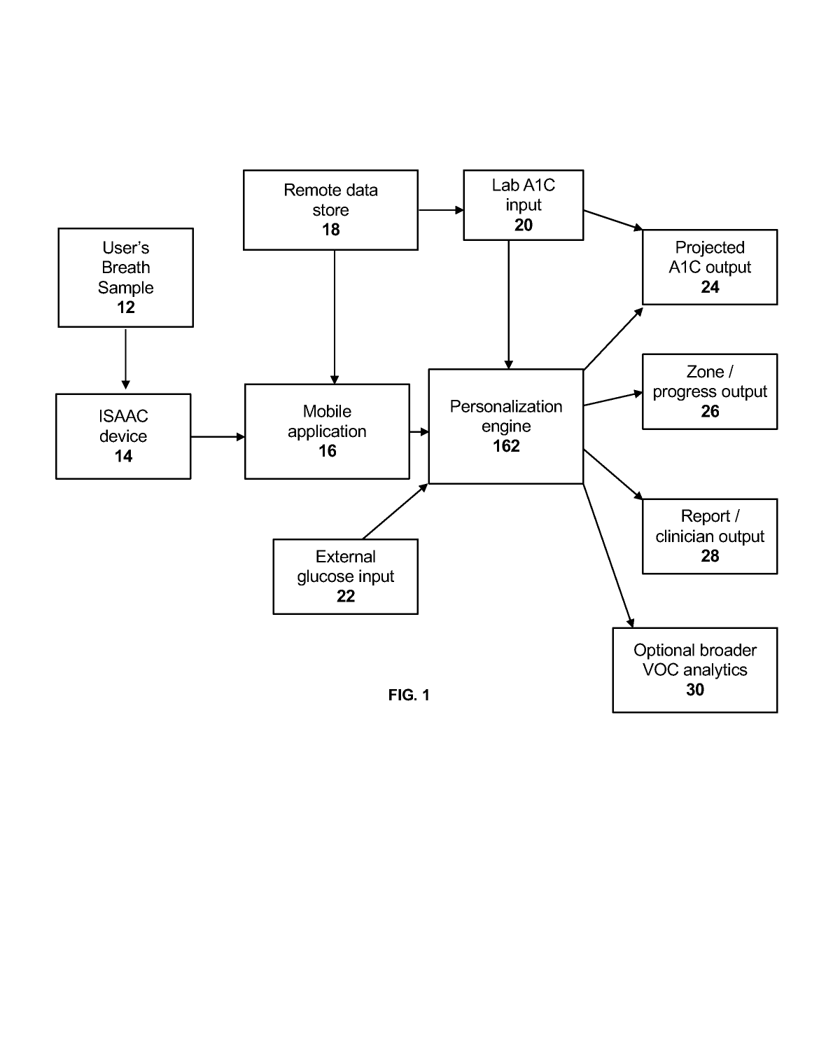

Referring to FIG. 1, an example breath-based glycemic monitoring system is configured to provide personalized, non-invasive monitoring of a user’s glycemic status over time. In the illustrated embodiment, a breath-sensing device 14, a user’s breath sample 12, a mobile application 16, a remote data store 18, a lab A1C input 20, an external glucose input 22, a projected A1C output 24, a zone / progress output 26, a report / clinician output 28, and an optional broader VOC analytics portion 30 are shown. The arrangement shown in FIG. 1 is illustrative rather than limiting, and the functions attributed to the various components may be distributed differently in other embodiments.

The breath-sensing device 14 is configured to acquire one or more user breath samples 12 and generate breath-related sensor data from those samples. In some embodiments, The breath-sensing 14 is a wearable device, such as a pendant-style device, although handheld, desktop, bedside, vehicle-mounted, or other form factors may also be used. The device may include one or more VOC sensors, one or more environmental sensors, control electronics, communication circuitry, and local memory. In some embodiments, The breath-sensing 14 performs at least partial on-device processing, such as collecting raw sensor signals, performing initial filtering, associating a timestamp with a measurement session, generating one or more quality indicators, or transmitting breath-session data to another component of the system for further analysis.

The mobile application 16 may operate on a smartphone, tablet, wearable companion device, dedicated controller, or other computing platform and may serve as a primary user interface for the system. In some embodiments, the mobile application 16 guides the user through onboarding, breath collection, sample timing, session completion, and review of results. The mobile application 16 may also present one or more prompts, reminders, notifications, trend summaries, notes interfaces, export controls, or clinician-sharing features. Communication between the breath-sensing device 14 and the mobile application 16 communication may occur using Bluetooth Low Energy, Wi-Fi, near-field communication, wired connection, or other suitable communication techniques. In some embodiments, the system may operate without wireless communication, or may operate without a wireless connection, and in other embodiments, certain communications are withheld until the wireless connection resumes.

The remote data store 18 may be provided as a cloud-based data repository, a network-accessible database, a secure server environment, or another local or remote storage architecture. In some embodiments, the remote data store 18 stores user profile information, baseline values, historical breath sessions, environmental metadata, derived metrics, model parameters, reports, or other information used by the system. In some implementations, some or all of the functionality of the remote data store 18 may instead be performed locally on the user’s mobile device or on The breath-sensing 14 itself. Accordingly, the storage architecture may be centralized, distributed, hybrid, temporary, persistent, or otherwise implemented in a manner suitable for the intended use environment.

The lab A1C input 20 may provide a baseline or anchor value for personalization of the system. In some embodiments, the lab A1C input 20 is based on a laboratory test result obtained at or near onboarding. The lab A1C input 20 may be entered manually by the user, received from a clinician, imported from a laboratory portal, retrieved from an electronic health record system, transferred from a third-party application, or otherwise supplied to the system. In certain embodiments, the system uses the lab A1C input 20 not as a mere display value, but as a reference point for initializing, calibrating, constraining, or refining a user-specific model that interprets breath-derived data over time.

The external glucose input 22 represents optional additional glycemic information that may be used to supplement or refine the personalized monitoring process. In some embodiments, the external glucose input 22 includes one or more finger-stick glucose values, continuous glucose monitor values, laboratory glucose values, estimated average glucose values, clinician-entered values, or imported data from third-party monitoring platforms. The external glucose input 22 may be used periodically, continuously, opportunistically, or only when the user or system seeks additional confirmation. In some embodiments, the external glucose input 22 is not required for routine operation after onboarding, but may improve personalization, anomaly handling, confidence scoring, or clinical interpretation.

The system may further include a personalization / data-fusion engine 162 configured to process breath-derived information together with the lab A1C input 20, the external glucose input 22, and optionally one or more historical or contextual variables. In some embodiments, the personalization / data-fusion engine 162 is implemented at least in part within the mobile application 16. In other embodiments, the personalization / data-fusion engine 162 is implemented in a remote computing environment associated with the remote data store 18, in The breath-sensing 14, or in a hybrid architecture in which different portions of the logic are executed in different locations. The personalization / data-fusion engine 162 may generate or update one or more user-specific relationships between breath-derived VOC information and one or more glycemic indicators so that the system becomes more individualized with repeated use.

Using the collected and personalized data, the system may generate one or more outputs for the user or for authorized third parties. In the embodiment of FIG. 1, such outputs include the projected A1C output 24, the zone / progress output 26, and the report / clinician output 28. The projected A1C output 24 may represent an estimated, inferred, modeled, projected, or trend-related indicator associated with longer-term glycemic status. In some embodiments, the projected A1C output 24 is derived from repeated breath sessions and a personalized model anchored by the lab A1C input 20, rather than constituting a direct laboratory measurement. The zone / progress output 26 may present the user’s current direction, status, trend, or relative performance using a simplified output format such as a score, color range, category, rank, arrow, icon, textual label, or other progress-oriented presentation. The report / clinician output 28 may include a more detailed summary of historical sessions, trend changes, notes, thresholds, anomalies, cross-checks, or recommendations for follow-up.

In some embodiments, the system is configured for longitudinal operation rather than one-time testing. Repeated breath sessions acquired using The breath-sensing 14 may be accumulated over time and evaluated with respect to a rolling history, thereby allowing the system to present daily, periodic, or event-driven feedback to a user. Such feedback may help the user understand whether current behaviors appear to be moving long-term glycemic status in a favorable direction, an unfavorable direction, or a stable direction. The system may therefore emphasize behavior-linked progress and longitudinal trend awareness, rather than relying solely on isolated snapshots.

The system may also support anomaly detection, threshold evaluation, confidence assessment, or confirmatory workflows. For example, when a breath session appears inconsistent with prior history, is associated with poor sample quality, falls outside an expected range, or indicates a concerning trend, the system may flag the session, reduce confidence, request a repeat breath session, or prompt the user to obtain a confirmatory reading or clinical test. Such confirmatory workflows may use the external glucose input 22 or another source of verification. In this manner, the system may provide user-friendly outputs while still preserving pathways for additional validation when appropriate.

The optional broader VOC analytics portion 30 indicates that the disclosed architecture may also support broader VOC-analysis workflows beyond glycemic monitoring. In some embodiments, the same or similar sensing infrastructure, data handling architecture, model framework, or reporting environment may be adapted for other VOC-based screening, classification, or condition-monitoring applications. Accordingly, while the present application emphasizes personalized glycemic monitoring and projected A1C-related outputs, the disclosed system architecture may also be extended to other analyte-driven or VOC-driven use cases.

Although FIG. 1 illustrates the various components as distinct blocks, that illustration is intended to show functional relationships rather than a required hardware partition. Any one or more of the mobile application 16, remote data store 18, personalization / data-fusion engine 162, and associated output functions may be implemented in a single device, in multiple cooperating devices, or in a partially distributed environment. Similarly, certain functions described as being performed remotely may instead be performed on-device, and certain functions described as being performed on-device may instead be performed by a paired mobile device or by remote computing resources. The disclosed subject matter therefore encompasses centralized, distributed, hybrid, cloud-assisted, and edge-computing implementations.

In operation, the system of FIG. 1 provides an overall framework in which breath-derived measurements from The breath-sensing 14 are combined with a user-specific baseline and, in some cases, optional external glycemic information so as to generate recurring, personalized outputs that are more meaningful than non-personalized, one-time estimates. The remaining figures describe example device structures, onboarding workflows, daily monitoring workflows, user-interface arrangements, and data-fusion embodiments that may be used within the overall system environment shown in FIG. 1.

### Example Pendant Device Exterior and Internal Construction

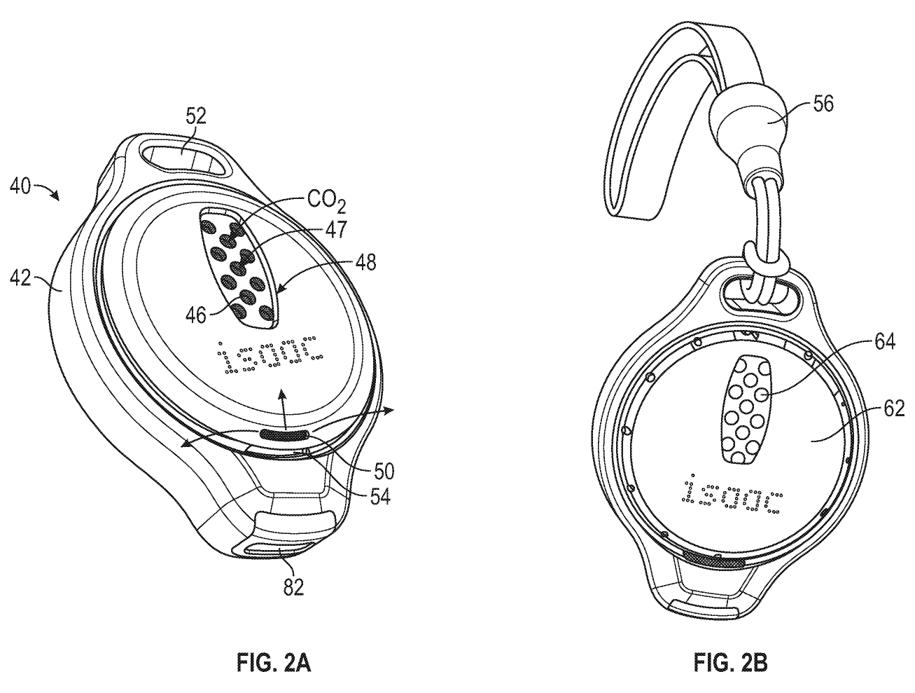

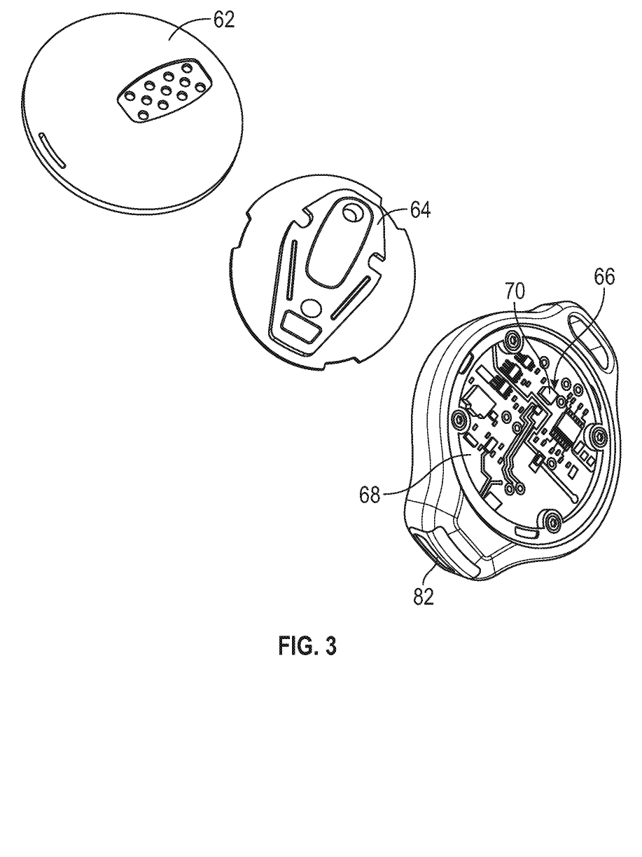

Referring to FIGS. 2A, 2B, and 3, the disclosed system may employ a compact breath-sensing device 40 configured for repeated everyday use. In the illustrated embodiment, the device 40 is generally pendant-shaped and sized to be worn, carried, or held conveniently by a user during ordinary daily activity. The device 40 may be implemented as a wearable pendant, a lanyard-mounted unit, a clipped unit, or a similarly portable form factor intended to support frequent breath sessions without the inconvenience of conventional invasive monitoring tools.

As shown in FIGS. 2A and 2B, the device 40 may include a housing or body 42 that encloses sensing, control, and communication components. The housing or body 42 may have a generally flattened, rounded, or ergonomic outer profile that is comfortable for repeated handling and suitable for use as a pendant-style product. In some embodiments, the housing or body 42 is formed from metal, polymer, composite material, or a combination thereof. For example, the housing may include an aluminum outer cover, a molded polymer support structure, or another enclosure configuration selected for durability, appearance, manufacturability, and resistance to environmental exposure.

The front portion of the device may include a breath inlet region 46. In the illustrated embodiment, the breath inlet region 46 is associated with an aperture array 48 arranged on a forward face of the device. The aperture array 48 may include a plurality of openings sized and positioned to admit a user’s exhaled breath 47 while helping protect internal components from larger contaminants. The specific number, size, spacing, and geometry of the openings may vary from one embodiment to another. In some implementations, the aperture array is elongated or vertically oriented, although other configurations may also be used.

The device may further include a lower opening or gas outlet port 50. In some embodiments, the lower opening or gas outlet port 50 is positioned below the breath inlet region 46 so that exhaled breath can pass through the device along a defined or semi-defined internal flow path. Such a flow path may help expose the sensing region to a fresh sample and may also help evacuate residual gas from a prior session. In some embodiments, the lower opening or gas outlet port 50 functions primarily as a vent or outlet. In other embodiments, the lower opening may also support pressure equalization, passive purging, or related airflow management functions.

An attachment loop 52 may be provided to support wearable use. The attachment loop 52 may be formed integrally with the housing or may be provided as a separate structural feature coupled to the housing. The attachment loop 52 may receive items such as a lanyard 56, clasp, ring, cord, chain, clip, or other securement member so that the device may be worn around the neck, coupled to clothing or a bag, or otherwise carried in a ready-to-use manner. Although pendant-style use is presently preferred, the disclosed subject matter is not limited to any particular securement technique.

A status indicator 54 may be positioned on the outer surface of the device to provide user feedback. In some embodiments, the status indicator 54 is a light source such as an LED or multi-state visual indicator. The status indicator 54 may communicate, for example, readiness, active sampling, successful session completion, pairing state, charging state, fault conditions, low-power state, alert conditions, or other operating information. The manner of presentation may include different colors, flashing patterns, intensities, icons, or other visual cues.

Referring now to FIG. 3, an exploded internal component view is shown. In the illustrated embodiment, the device includes a front cover 62, a dust filter 64, a hydrophobic barrier 66, a printed circuit board 68, a sensor assembly 70, and a data / power port 82. These components are arranged within the enclosure so that breath entering through the breath inlet region can be conditioned and presented to the sensing components in a controlled manner. The exploded arrangement is illustrative and is not intended to require a particular assembly order, mounting method, or mechanical stack-up. Not shown is an internal battery, which may be provided as is known in the art. In some embodiments, the internal battery may be replaceable and / or may be charged via power port 82.

The front cover 62 may define or help support the visible front-facing portion of the device. In some embodiments, the front cover 62 includes or aligns with the breath inlet region 46 and aperture array 48. The front cover 62 may also provide a protective and aesthetic outer surface. In certain embodiments, the front cover 62 is removable to facilitate assembly, service, filter replacement, or access to internal components. In other embodiments, the front cover 62 is permanently secured to the remainder of the housing.

The dust filter 64 may be positioned inward of the front cover 62 and may help reduce ingress of lint, dust, particulate matter, or other contaminants. The hydrophobic barrier 66 may be positioned behind the dust filter 64 and may help reduce the effect of liquid droplets, condensation, saliva aerosols, or moisture exposure while still allowing gas-phase components of the breath sample to reach the sensing region. In some embodiments, the dust filter 64 and hydrophobic barrier 66 are separate layers. In other embodiments, their functions may be combined in a single filtering structure or membrane assembly.

The printed circuit board 68 may support the at least one sensor assembly 70 together with associated electronics. In some embodiments, the printed circuit board 68 carries signal-conditioning components, processing components, wireless communication components, memory, power-management circuitry, charging circuitry, and connector structures. The printed circuit board 68 may also carry one or more heaters, control circuits, or environmental sensing circuits used in connection with the breath analysis process. The layout shown in FIG. 3 is schematic in the sense that individual components may be rearranged, combined, or separated in different commercial embodiments.

The at least one sensor assembly 70 is configured to generate breath-related sensing information responsive to a user’s exhaled breath. In some embodiments, the at least one sensor assembly 70 includes a VOC sensor configured to detect one or more breath-borne compounds correlated with glucose-related metabolic status. In certain embodiments, the at least one sensor assembly 70 further includes humidity sensing and temperature sensing capability. Although FIG. 3 may depict the sensing functionality as a single component for simplicity, the VOC sensor, humidity sensor, and temperature sensor may be provided as separate components, as a partially integrated module, or as a fully integrated multi-function sensing package.

In use, breath entering the breath inlet region 46 may pass through the aperture array 48, through or across the dust filter 64 and hydrophobic barrier 66, and toward the at least one sensor assembly 70 mounted relative to the printed circuit board 68. The resulting sensor information may then be processed locally on the device, transmitted to a paired mobile application, or both. In some embodiments, the humidity and temperature information associated with the breath session is used to assess sample quality, normalize a reading, reject an anomalous session, or otherwise improve interpretation of the breath-derived signal.

The data / power port 82 may support electrical charging, wired data transfer, firmware update operations, diagnostics, factory programming, or combinations thereof. In some embodiments, the device includes an internal rechargeable power source that is charged through the data / power port 82. In other embodiments, the port may additionally or alternatively provide a direct wired communication path to a host device, a service fixture, or a docking accessory. The precise port type may vary and may include, for example, a wired connector, charging pads, pogo contacts, or another suitable electrical interface.

In some embodiments, internal non-sensing regions of the device may be protected by coatings, conformal layers, sealing materials, or other protective structures to improve durability in the presence of ambient humidity, sweat, dust, and repeated consumer handling. Likewise, one or more gaskets, adhesive layers, retainers, or support frames may be used to help hold the internal stack in position and to maintain alignment between the breath inlet region 46 and the sensor assembly 70. The disclosed subject matter is not limited to any specific sealing or packaging technique.

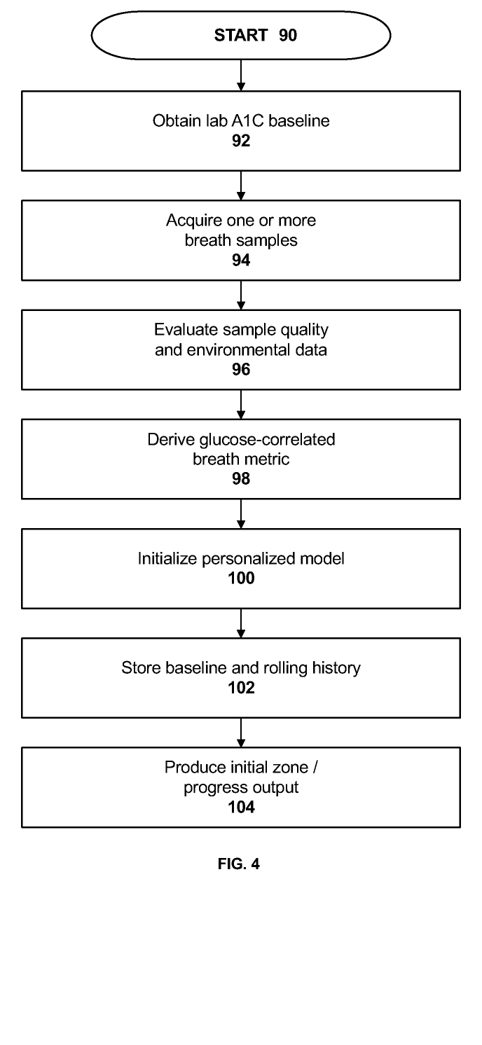

The device construction described with respect to FIGS. 2A, 2B, and 3 therefore provides a compact breath-sensing architecture in which external breath-entry features, internal filtering features, and sensing electronics are integrated into a wearable or portable enclosure suitable for repeated daily use. The operation of the device during onboarding and personalization is described next with respect to FIG. 4.

### Example Onboarding and Personalization Process

Referring to FIG. 4, an example onboarding and personalization process 90 is shown. In general, the onboarding and personalization process 90 establishes an initial user-specific framework for interpreting breath-derived measurements so that later outputs are based on a personalized baseline rather than on a purely generalized population model. In the illustrated embodiment, the process 90 includes obtaining a lab A1C baseline 92, acquiring breath samples 94, performing sample-quality and environmental evaluation 96, deriving a glucose-correlated breath metric 98, initializing a personalized model 100, storing baseline and rolling history 102, and providing an initial zone or progress output 104. Although these operations are shown in a particular order for ease of explanation, the sequence may be rearranged, repeated, subdivided, combined, or supplemented in other embodiments.

The onboarding and personalization process 90 may be performed using The breath-sensing 14 together with the mobile application 16 and, in some embodiments, the remote data store 18 and the personalization / data-fusion engine 162 described with respect to FIG. 1. In one example implementation, a user first pairs The breath-sensing 14 with the mobile application 16, creates or activates a user account, and is guided through an onboarding sequence designed to collect initial information needed to personalize later analysis. The onboarding sequence may include explanatory screens, consent screens, instructions for breath collection, reminders regarding timing, and prompts for entry or import of baseline information.

At step 92, the system obtains a lab A1C baseline. In preferred embodiments, the lab A1C baseline is a recent laboratory-derived A1C value associated with the user. The value may be entered manually by the user, entered by a clinician, imported from a laboratory portal, received from an electronic health record source, retrieved from an application programming interface, or otherwise supplied to the system. The system may also store associated metadata such as the date of the laboratory test, the source of the value, units, confidence information, or whether the value was verified. In some embodiments, the system may prompt the user to confirm that the entered value corresponds to a recent clinical test rather than to a device-generated estimate.

In some implementations, the system may evaluate the lab A1C baseline for suitability before using it to personalize the user profile. For example, the system may check whether the lab value is sufficiently recent, falls within an expected range, or appears complete and internally consistent. If the supplied value does not satisfy one or more criteria, the system may request confirmation, request a newer value, defer use of the value, or initiate a provisional operating mode until an updated clinical baseline is available. In this manner, the system may distinguish between a reliable calibration anchor and a merely historical or uncertain value.

The lab A1C baseline obtained at step 92 is not necessarily treated as a one-time static display item. Rather, in certain embodiments, it is used as an anchor for initializing a user-specific relationship between breath-derived information and longer-term glycemic interpretation. This distinction may be significant in embodiments where the system is intended to provide day-to-day motivational or directional information rather than claiming to generate an immediate out-of-the-box clinical A1C result solely from a single breath sample.

At step 94, the system acquires breath samples from the user. In some embodiments, the user is guided through one or more onboarding breath sessions using the mobile application 16 and The breath-sensing 14. The guidance may include one or more prompts relating to device placement, device readiness, inhalation timing, exhalation timing, sample duration, repeated sampling, or session completion. In some embodiments, the onboarding process includes multiple breath samples collected during a single session, while in other embodiments multiple onboarding sessions may be collected over a period of time in order to establish a more stable baseline. The system may instruct the user to provide a steady exhalation, a defined exhalation interval, multiple exhalations, or another controlled breath pattern suitable for the sensing architecture.

In some embodiments, the breath samples collected at step 94 are associated with contextual information that may improve interpretation. For example, the system may collect or infer a session timestamp, time of day, elapsed time since a prior measurement, elapsed time since eating, medication timing, exercise timing, stress indicators, sleep-related information, user notes, or other contextual metadata. Some of this information may be entered manually by the user, while other information may be determined automatically or inferred from connected systems. The system may use such information during personalization, filtering, anomaly detection, or later reporting.

At step 96, the system performs sample-quality and environmental evaluation. This step may use information from one or more sensors on The breath-sensing 14, such as humidity sensing, temperature sensing, flow-related indicators, timing information, sensor stabilization characteristics, or other quality-related information. The system may evaluate whether the breath sample appears complete, whether the device was used properly, whether environmental conditions fall within acceptable ranges, whether excessive moisture or contamination may be present, whether the session deviates from an expected pattern, or whether the sensing response appears unstable or anomalous.

If the sample-quality and environmental evaluation 96 indicates that a breath sample is unsuitable or has low confidence, the system may reject the sample, reduce its weighting, prompt the user to repeat the sampling step, or store the sample with an associated quality flag. In some embodiments, the system distinguishes between fully accepted onboarding samples, partially accepted onboarding samples, and rejected onboarding samples. In other embodiments, the system may require a threshold number of acceptable onboarding samples before allowing initialization of a personalized model. This can help reduce the influence of poor technique, inadequate breath capture, or adverse ambient conditions during the personalization phase.

At step 98, the system derives a glucose-correlated breath metric from the accepted breath sample information. The glucose-correlated breath metric may be based on one or more sensor outputs associated with VOC detection, and may optionally incorporate humidity, temperature, or other contextual adjustments. In some embodiments, the glucose-correlated breath metric may be derived from one or more raw sensor values, normalized sensor values, temporal response characteristics, peak values, area-under-curve values, response slopes, ratios among sensed features, filtered signals, or composite feature vectors. The particular mathematical form of the glucose-correlated breath metric may vary depending on the sensor type, calibration strategy, and personalization approach.

In certain embodiments, the glucose-correlated breath metric is not limited to a single scalar value. Instead, it may be a structured feature set, latent representation, classifier input, ranked signature set, or other intermediate representation suitable for use by the personalization / data-fusion engine 162. Likewise, in some implementations, derivation of the glucose-correlated breath metric may occur partly on The breath-sensing 14, partly in the mobile application 16, partly within the remote data store 18 or associated computing environment, or in any suitable combination thereof.

At step 100, the system initializes a personalized model. In some embodiments, the personalized model uses the lab A1C baseline obtained at step 92 together with the glucose-correlated breath metric derived at step 98 to establish an initial personalized mapping, calibration relationship, weighting structure, or interpretation profile for the user. The personalized model may take many forms, including a regression model, a rules-based model, a classifier, a probabilistic model, a neural-network-based model, a hybrid model, or another adaptive framework. The system may generate model coefficients, thresholds, normalization factors, confidence values, or other user-specific parameters during this initialization step.

In some embodiments, initializing the personalized model 100 includes determining an initial baseline state for the user and defining how subsequent breath-derived sessions will be compared against that state. For example, later sessions may be interpreted relative to the baseline to determine whether the user's inferred glycemic trajectory appears to be improving, worsening, or remaining stable. In some implementations, the personalized model may also incorporate one or more optional contextual variables, such as user profile information, historical medical information, device-specific calibration data, or session-quality measures.

The personalized model initialized at step 100 may be regarded as preliminary, semi-final, or adaptive depending on the number and quality of onboarding samples obtained. In some embodiments, the system may create an initial personalized model after a first acceptable onboarding session and then refine that model as additional acceptable onboarding sessions are collected. In other embodiments, the system may refrain from providing certain outputs until multiple acceptable sessions have been obtained. Accordingly, "initialization" does not necessarily mean that the model is fixed; rather, it may represent the beginning of an ongoing personalization process.

At step 102, the system stores baseline and rolling history information. This stored information may include the lab A1C baseline, timestamps, accepted breath sessions, rejected or low-confidence sessions, normalized sensor data, glucose-correlated breath metrics, initial model parameters, quality indicators, environmental values, contextual notes, and related metadata. Storage may occur locally on the mobile application 16, remotely in the remote data store 18, on The breath-sensing 14, or in a distributed combination of such locations. In some embodiments, the system uses the stored information to support later recalibration, trend analysis, report generation, clinician review, audit trails, or synchronization across devices.

The rolling history established at step 102 may begin during onboarding rather than only after onboarding is complete. That is, the system may treat accepted onboarding measurements as the first entries in a longitudinal record from which later trends, scores, and projected A1C-related indicators are developed. In this way, the onboarding process 90 may serve not merely as setup, but as the beginning of the user's personalized monitoring journey.

At step 104, the system may provide an initial zone or progress output. In some embodiments, this initial output is designed to communicate an early personalized status in a simplified and user-friendly manner. For example, the output may be presented as a zone, score, band, category, trend indicator, directional symbol, color-coded status, or similar progress-oriented display. The initial output may indicate, for example, a starting status, an initial risk grouping, a baseline-relative condition, or an early estimate of whether the user's day-to-day pattern appears favorable or unfavorable relative to the established anchor.

In some implementations, the initial zone or progress output 104 is accompanied by explanatory content that informs the user that the system is entering a personalization phase and that confidence, stability, or precision may improve with repeated use. The system may also recommend continued daily or periodic sessions so that later outputs become more individualized and trend-aware. In other embodiments, the initial output may be intentionally conservative and may emphasize directionality rather than precise numerical prediction.

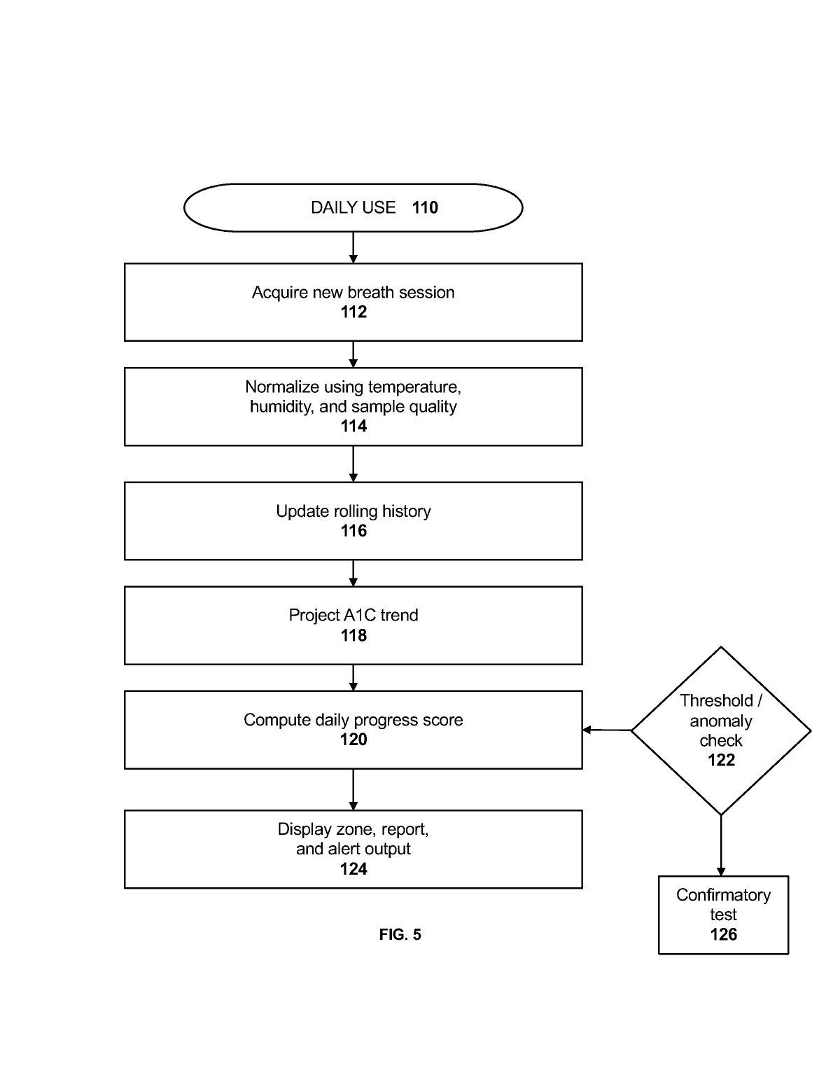

The onboarding and personalization process 90 therefore provides a technical and user-facing framework for anchoring later breath-based interpretation to a known laboratory reference while also screening for sample quality, deriving breath-related metrics, and building an individualized baseline that can be refined over time. Once the user has completed onboarding and the personalized model has been initialized, the system may proceed to repeated day-to-day operation. An example daily monitoring process is described next with respect to FIG. 5.

### Example Daily Monitoring and Longitudinal Update Process

Referring to FIG. 5, an example daily monitoring process 110 is shown. In general, the daily monitoring process 110 is performed after onboarding and personalization have been established, and is configured to provide recurring user-facing feedback based on breath-derived data acquired over time. In the illustrated embodiment, the daily monitoring process 110 includes acquiring a new breath session 112, normalizing the session using temperature, humidity, and sample quality 114, updating rolling history 116, projecting an A1C trend 118, computing a daily progress score 120, performing a threshold / anomaly check 122, displaying zone, report, and alert output 124, and optionally issuing a confirmatory test prompt 126. Although FIG. 5 presents these operations in a particular order, the sequence may be varied in other embodiments, and one or more operations may be repeated, omitted, combined, or performed in parallel.

The daily monitoring process 110 may be initiated manually by the user, automatically in response to a reminder, or in response to another event such as a scheduled monitoring time, a requested follow-up session, or detection of a prior low-confidence session. In some embodiments, the system encourages repeated use on a daily basis so that the personalized model continues to accumulate information and provide directional insight regarding the user's glycemic trajectory. In other embodiments, multiple sessions may be collected per day, such as before meals, after meals, before sleep, after exercise, or at other times that may be useful for characterizing trends or excursions.

At step 112, the system acquires a new breath session. The new breath session may include one breath sample or multiple breath samples collected during a single user interaction. In some embodiments, the mobile application 16 instructs the user regarding device positioning, readiness, exhalation timing, exhalation duration, or repetition of the breath sequence. The breath-sensing 14 may collect raw or partially processed sensor data during the session and may associate the session with time information, device status, and other contextual metadata. In certain embodiments, the system may accept multiple exhalations and select a preferred sample, combine multiple samples, or derive a representative session value from more than one sensing event.

The breath session acquired at step 112 may also be associated with contextual information that can assist later interpretation. For example, the system may store a timestamp, time since last session, time since last meal, medication information, user-entered notes, exercise information, sleep-related information, or other context. In some embodiments, the system may infer at least part of this context from connected devices, prior records, or user behavior patterns. In other embodiments, contextual information is optional and may be absent for some sessions.

At step 114, the system normalizes the new session using temperature, humidity, and sample quality. This normalization step may use information from the at least one sensor 70, which may be one or more of a VOC sensor, humidity sensor, or temperature sensor, and associated processing components of the device. In some embodiments, normalization includes compensation for humidity level, breath temperature, ambient temperature, ambient humidity, device warm-up state, sensor stabilization behavior, baseline drift, or other variables that may influence interpretation of a breath-derived signal. The normalization step may also include evaluating whether the user provided a sufficiently complete or consistent breath sample.

Sample-quality handling at step 114 may include acceptance, rejection, weighting, or flagging of the session. For example, if the breath session appears too short, too noisy, too moisture-heavy, incomplete, or otherwise inconsistent with expected device behavior, the system may reject the session, ask the user to repeat the session, or store the session with a reduced-confidence indicator. In some embodiments, normalization and sample-quality assessment are tightly coupled, such that the system both corrects for routine variation and separately determines whether the session is sufficiently reliable for inclusion in longitudinal analysis.

After normalization, the system may derive a normalized breath-session representation suitable for longitudinal comparison. In some embodiments, that representation may include one or more normalized sensor values, peak responses, temporal features, filtered responses, ratios, or composite features derived from the breath session. The normalized representation may then be compared against the user-specific model established during onboarding and, in some embodiments, updated over time through repeated use.

At step 116, the system updates rolling history. The rolling history may include accepted daily sessions together with associated timestamps, normalized values, quality indicators, contextual metadata, prior projected A1C-related indicators, prior scores, and related information. In some embodiments, updating rolling history includes appending the current session to a chronological record and recalculating one or more rolling-window values. In other embodiments, updating rolling history may include weighting recent sessions more heavily than older sessions, excluding low-confidence sessions, smoothing short-term variability, or maintaining separate histories for different usage contexts, such as fasting sessions versus post-meal sessions.

The rolling history may be maintained over any suitable time horizon. In some embodiments, the system stores a short-term history for daily scoring, an intermediate-term history for trend identification, and a longer-term history used in projecting A1C-related change over time. The precise time windows, weighting functions, and smoothing methods may vary across embodiments. What is important is that the system is not limited to a single isolated breath reading, but instead interprets new sessions within an individualized temporal context.

At step 118, the system projects an A1C trend. In some embodiments, the projected A1C trend is derived from the rolling history together with the personalized model initialized using the lab A1C baseline. The projected A1C trend may represent a modeled estimate, an inferred trend direction, a projected change indicator, a future-state approximation, or another longer-horizon glycemic indicator associated with cumulative recent behavior. In preferred embodiments, the projected A1C trend is not presented as a substitute for laboratory testing, but rather as a personalized longitudinal indicator intended to help the user understand the likely direction of longer-term glycemic control.

The projection performed at step 118 may be updated after each accepted breath session or after a defined number of sessions. In some embodiments, the projection uses recency weighting, rolling averaging, trend fitting, or model-based inference. In certain implementations, the projected A1C trend may be represented numerically. In other implementations, the projection may be translated into a category, band, zone, or directional output that is easier for the user to interpret. The projected A1C trend may also be accompanied by a confidence value, quality indicator, or explanatory message, especially in cases where available data is sparse or inconsistent.

At step 120, the system computes a daily progress score. The daily progress score may be a user-facing output intended to summarize whether the user's current pattern appears favorable, unfavorable, or neutral relative to the user's personalized baseline and recent trend history. In some embodiments, the daily progress score is expressed as a numerical score. In other embodiments, it is expressed as a zone, color category, label, rank, icon, arrow, or similar simplified presentation. The daily progress score may reflect not only the current normalized breath session, but also how that session compares with recent sessions and with the projected A1C trend.

In some embodiments, the daily progress score is deliberately designed as a motivational output rather than a clinical diagnosis. For example, the score may help the user understand that repeated high-glucose-associated events or unfavorable patterns appear to be moving the user's longer-term trajectory in an undesirable direction, while sustained favorable patterns may be improving that trajectory. The score may therefore be particularly useful for encouraging consistent daily engagement and behavior modification, even where the system does not purport to deliver a laboratory-grade A1C value from a single breath test.

At step 122, the system performs a threshold / anomaly check. This check may examine the current session, the updated rolling history, the projected A1C trend, the daily progress score, or any combination thereof. In some embodiments, the threshold / anomaly check identifies sudden deviations, concerning trends, unexpected jumps, low-confidence sessions, contradictions with prior history, prolonged deterioration, or values that fall outside expected limits. The system may also evaluate whether the observed result is inconsistent with available contextual data or with optional external glucose information, where such information is available.

The threshold / anomaly check 122 may produce different system responses depending on the nature of the condition detected. For example, the system may merely flag a result for informational purposes, may display a cautionary message, may reduce confidence in the result, may suppress a score pending additional data, or may request a repeat breath session. In more pronounced cases, the system may generate an alert or recommend that the user obtain a confirmatory measurement or clinical follow-up. The anomaly logic may therefore function as a safety and quality layer in addition to its role in improving model robustness.

At step 124, the system displays zone, report, and alert output. In some embodiments, the displayed output includes a current zone or progress presentation, a projected A1C-related tile or trend display, and one or more alerts, notes, or explanatory indicators. The output may be shown on the mobile application 16, on a paired device, on The breath-sensing 14 itself in simplified form, or through another user interface. The displayed information may include current-session results, historical summaries, directional changes, quality flags, educational prompts, reminders, or other information designed to help the user understand the significance of the new session.

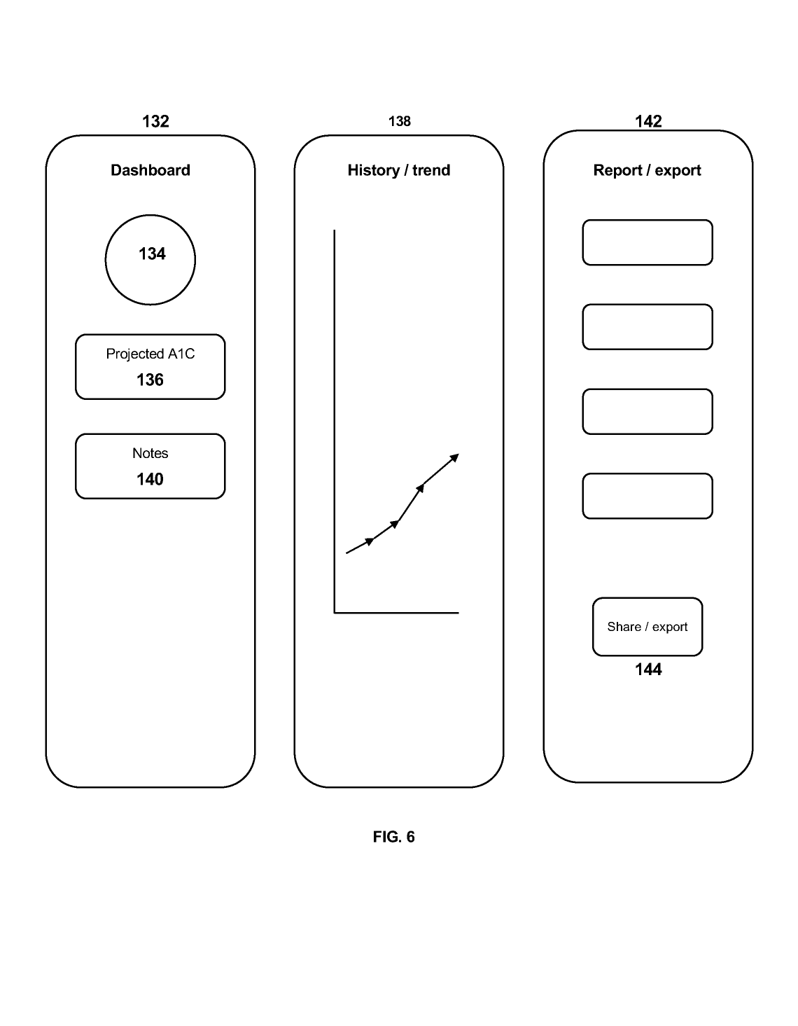

The report portion of step 124 may support more detailed review than the simplified zone or score presentation. For example, the system may prepare a session summary, a recent-history summary, a projected trend summary, or a shareable report for later export or clinician review. In some embodiments, the user may also be offered an opportunity to enter notes associated with the session, such as food intake, exercise, symptoms, stress, or medication changes. Further examples of user-interface and reporting structures are described below with respect to FIG. 6.

At step 126, the system may issue a confirmatory test prompt. The confirmatory test prompt may be triggered automatically in response to the threshold / anomaly check 122, in response to low confidence, or in response to a user request. In some embodiments, the confirmatory test prompt instructs the user to repeat the breath session. In other embodiments, it may recommend a finger-stick reading, consultation of a continuous glucose monitor, entry of an external glucose value, or obtaining a laboratory or clinical test. The confirmatory test prompt may therefore serve both as a user guidance tool and as a mechanism for improving confidence in situations where the current breath-derived result is uncertain or concerning.

In some embodiments, information obtained in response to the confirmatory test prompt 126 may be stored and later used to refine the user's personalized model. For example, a confirmatory finger-stick value, CGM value, or laboratory result may be associated with the corresponding breath session and used as an additional calibration or cross-check data point. In this manner, the daily monitoring process 110 may not only generate recurring outputs, but may also improve personalization over time when additional information becomes available.

The daily monitoring process 110 thus provides a longitudinal workflow in which each accepted breath session contributes to an evolving, user-specific understanding of glycemic trajectory. By combining repeated breath-derived measurements, normalization, rolling-history maintenance, projected A1C-related trend generation, and simplified daily progress output, the system may provide a user with frequent and actionable feedback while still preserving anomaly handling and confirmatory pathways. Example user-interface and reporting implementations for presenting these outputs are described next with respect to FIG. 6.

### Example User Interface, Notes, Reporting, and Export Features

Referring to FIG. 6, an example user-interface / reporting architecture is shown. In general, the user-interface / reporting architecture provides a set of visual and interactive features through which the system communicates breath-session results, longitudinal trend information, projected longer-term glycemic indicators, notes, alerts, and shareable reporting content to a user and, in some embodiments, to an authorized clinician or other recipient. In the illustrated embodiment, the architecture includes a dashboard view 132, a zone indicator 134, a projected A1C tile 136, a history / trend view 138, a notes control 140, a report / export view 142, and a share / export control 144. Although these elements are shown as distinct interface components for ease of illustration, any one or more of them may be combined, rearranged, omitted, or distributed across multiple screens in other embodiments.

The user-interface / reporting architecture may be implemented primarily through the mobile application 16 described above with respect to FIG. 1. In some embodiments, certain simplified outputs may also be provided on The breath-sensing 14 itself, on a smartwatch, on a tablet, or through another paired computing environment. The interface may be optimized for repeated daily use so that a user can quickly understand whether a newly completed breath session suggests a favorable, neutral, or unfavorable direction relative to the user’s personalized glycemic baseline and recent history.

The dashboard view 132 may serve as a primary landing screen for the user. In some embodiments, the dashboard view 132 presents a concise summary of the user’s current monitoring status, recent session results, current zone or progress position, projected longer-term trend, reminders, and prompts for the next breath session. The dashboard view 132 may further display device status, synchronization status, last completed session time, session quality indicators, or notifications relating to low-confidence sessions, repeat-session requests, or confirmatory prompts. In some implementations, the dashboard view 132 is configured to emphasize clarity and low cognitive burden so that the user can understand current status at a glance.

The zone indicator 134 may provide a simplified user-facing output representing the user’s present status or direction. In some embodiments, the zone indicator 134 is presented as a colored region, category, band, label, score range, or similar simplified visual output. The zone indicator 134 may indicate, for example, whether the user appears to be trending in a favorable direction, a cautionary direction, or an unfavorable direction relative to the user’s personalized baseline and recent rolling history. In some implementations, the zone indicator 134 is intended to motivate behavior change by making longer-term glycemic implications more intuitive than a raw technical value alone.

In certain embodiments, the zone indicator 134 is generated from the current breath session together with prior accepted sessions, quality weighting, and the user-specific model established during onboarding. Thus, the zone indicator 134 need not correspond to a direct laboratory value or to a single-session glucose reading. Instead, the zone indicator 134 may represent a synthesized status output that incorporates personalization and longitudinal context. In some implementations, the system may also display an explanatory note or icon adjacent to the zone indicator 134 to indicate confidence level, recent change, or whether the current status was influenced by a low-confidence or repeated session.

The projected A1C tile 136 may present a projected, modeled, inferred, or estimated longer-term glycemic indicator derived from repeated breath sessions and the personalized baseline established using the lab A1C input 20. In some embodiments, the projected A1C tile 136 is shown as a numeric value, a value range, a directional change indicator, or a graphical tile summarizing a trend in the user’s projected longer-term glycemic status. In preferred embodiments, the projected A1C tile 136 is not presented as a laboratory measurement, but rather as a user-specific longitudinal indicator intended to help the user understand the likely direction or magnitude of change over time.

The projected A1C tile 136 may also include contextual qualifiers. For example, the tile may display an upward trend, a downward trend, a stable trend, a confidence level, or a notation indicating that additional data may improve reliability. In some embodiments, the projected A1C tile 136 is updated after each accepted breath session. In other embodiments, it may be updated after a threshold number of sessions, after a defined interval, or when sufficient new information is available to support a meaningful update. The projected A1C tile 136 may also present a comparison to a prior period, thereby helping the user understand whether the recent trajectory appears to be improving or worsening.

The history / trend view 138 may provide a more detailed representation of the user’s longitudinal data. In some embodiments, the history / trend view 138 presents a graph, timeline, chart, calendar, rolling-window display, or other visualization showing past breath sessions, prior zone outputs, projected longer-term trends, anomalies, confirmatory events, or associated contextual markers. The history / trend view 138 may allow the user to review day-to-day or week-to-week changes and to identify how repeated behaviors may correlate with changes in the user’s monitored trajectory.

In some implementations, the history / trend view 138 supports selectable time ranges, such as daily, weekly, monthly, or custom intervals. The view may also allow filtering or labeling by context, such as fasting sessions, post-meal sessions, exercise-associated sessions, low-confidence sessions, or sessions associated with external glucose inputs. In this way, the history / trend view 138 may function not only as a visual record but also as a tool for helping the user recognize patterns and potential triggers.

The notes control 140 may enable the user to associate optional notes or tags with a session or with a period of time reflected in the history / trend view 138. In some embodiments, the notes control 140 permits entry of free-text comments, predefined tags, icons, or selectable event labels. Example note categories may include meal information, exercise, medication, stress, sleep, illness, symptoms, travel, alcohol intake, fasting state, or other contextual factors that may help the user or a clinician interpret the monitoring history. In some embodiments, the notes control 140 may also support voice entry, dictated notes, or suggested tags based on prior user behavior.

Information associated through the notes control 140 may be stored with session metadata and later reflected in the dashboard view 132, history / trend view 138, report / export view 142, or other system outputs. In some embodiments, the notes control 140 may assist the user in identifying correlations between lifestyle factors and progress trends. In other embodiments, the stored notes may aid a clinician in reviewing the user’s self-monitoring record.

The report / export view 142 may present a more comprehensive summary of the user’s monitoring history and current status than is shown in the dashboard view 132. In some embodiments, the report / export view 142 includes one or more summaries of accepted breath sessions, projected A1C-related trends, zone history, progress-score history, anomalies, quality flags, confirmatory prompts, external glucose cross-checks, and user-entered notes. The report / export view 142 may be formatted for direct user understanding, clinician review, remote consultation, health coaching, wellness tracking, or related uses.

In some implementations, the report / export view 142 may present both a high-level summary and a detailed section. The high-level summary may include a current status overview, current projected longer-term trend, and recent directionality. The detailed section may include timestamps, historical plots, session-by-session indicators, note summaries, model-confidence indicators, external-input comparisons, or other supporting content. In some embodiments, the system may generate a shareable report in response to a user request, in response to a scheduled reporting interval, or automatically upon detection of a threshold event, anomalous pattern, or confirmatory prompt condition.

The share / export control 144 may permit the user to transmit, save, or otherwise provide the contents of the report / export view 142 to another destination. In some embodiments, the share / export control 144 supports export as a portable document, image, structured data file, secure message, electronic mail attachment, application-level share action, or direct transmission to a clinician portal or other authorized destination. In certain embodiments, the share / export control 144 may allow the user to select a date range, choose whether notes or external-input information are included, or generate a summary-only version versus a more complete report.

In some embodiments, export operations associated with the share / export control 144 are governed by privacy and authorization settings. For example, the system may require user confirmation before transmission, may restrict export to authorized recipients, may redact certain personal information, or may log report-sharing events for later review. Although FIG. 6 illustrates a share / export control 144 as a discrete interface element, the corresponding functionality may also be implemented through menus, gestures, scheduled automations, or background synchronization processes.

The user-interface / reporting architecture may also incorporate quality and safety messaging. For example, if a session used in a displayed result had low confidence, if a result is based on limited historical data, or if a confirmatory reading is recommended, the interface may display a banner, icon, message, or other indication near the relevant output. Likewise, the interface may distinguish between routine motivational outputs and conditions warranting closer follow-up. In some embodiments, the interface may clearly communicate that projected longer-term indicators are derived from a personalized breath-based model and are not direct substitutes for laboratory testing.

In some embodiments, the architecture supports adaptive presentation styles. A first user may prefer a highly simplified display centered on the zone indicator 134 and projected A1C tile 136, while another user or clinician may prefer detailed trends, notes, and export-ready reports. Accordingly, the system may permit configuration of display density, terminology, alert style, and reporting granularity. The same underlying monitoring data may therefore be rendered differently depending on the intended audience or use context.

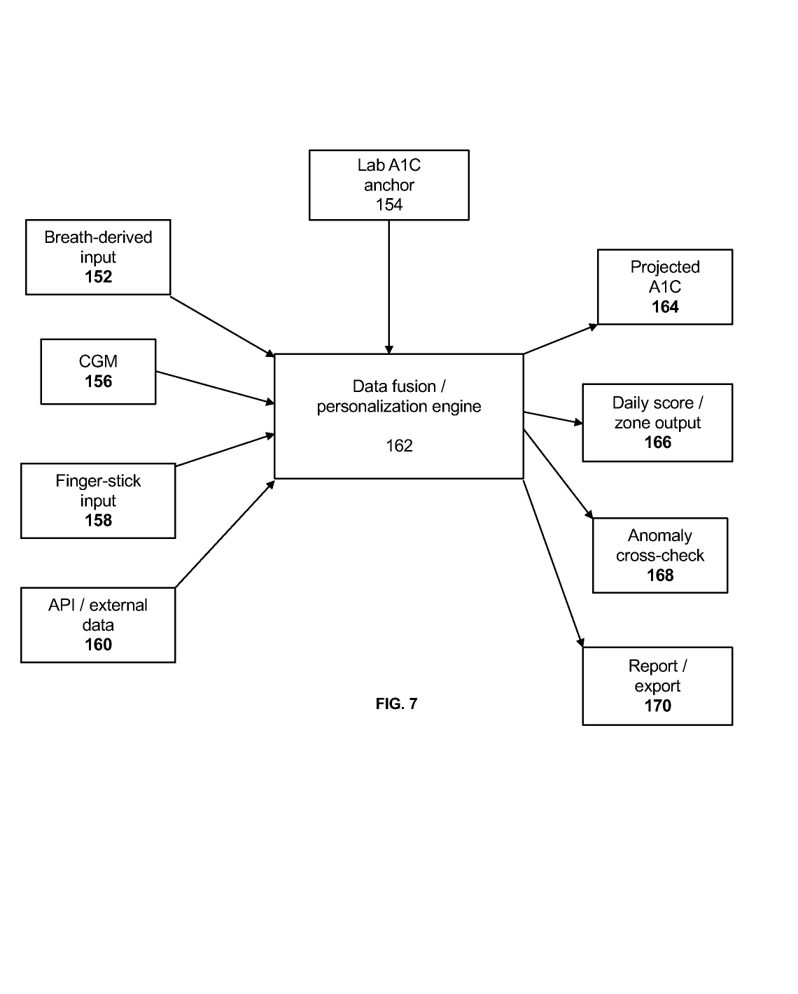

The user-interface / reporting architecture shown in FIG. 6 accordingly provides a practical mechanism for translating breath-derived monitoring results into outputs that are understandable, motivating, and clinically useful. By combining simplified status displays, longer-term projection tiles, longitudinal trend views, contextual note capture, and exportable report structures, the system may improve daily user engagement while also supporting more detailed review and communication when needed. Additional integration of breath-derived information with laboratory and external data sources is described next with respect to FIG. 7.

### Example Data-Fusion Architecture and Optional External Inputs

Referring to FIG. 7, an example data-fusion architecture is shown. In general, the data-fusion architecture permits the system to combine breath-derived information with one or more additional data sources so as to improve personalization, cross-check results, refine projected longer-term glycemic indicators, and support richer reporting. In the illustrated embodiment, the data-fusion architecture includes breath-derived input 152, a lab A1C anchor 154, a CGM input 156, a finger-stick input 158, an API / external data input 160, a personalization / data-fusion engine 162, a projected A1C output 164, a daily score / zone output 166, an anomaly cross-check 168, and a report / export output 170. The arrangement shown in FIG. 7 is illustrative rather than limiting, and one or more of the inputs or outputs may be omitted, combined, or supplemented in other embodiments.

The data-fusion architecture may be employed in a variety of operating modes. In some embodiments, the system relies primarily on breath-derived input 152 after onboarding and uses additional inputs only occasionally or when available. In other embodiments, the system regularly incorporates one or more additional data streams to refine personalization over time. Thus, FIG. 7 should be understood as showing an optional and extensible architecture in which the disclosed breath-based monitoring system may operate as a largely self-contained daily-use platform or as a hybrid platform that benefits from periodic confirmation, cross-calibration, or supplemental context.

The breath-derived input 152 may include one or more normalized breath-session outputs obtained from The breath-sensing 14 and associated processing described above with respect to FIGS. 1-5. In some embodiments, the breath-derived input 152 includes raw or partially processed sensor data, normalized feature values, glucose-correlated breath metrics, session-quality indicators, environmental values, timestamps, contextual metadata, or combinations thereof. The breath-derived input 152 may be generated at repeated intervals and may serve as the principal recurring input used for daily monitoring and user engagement.

The lab A1C anchor 154 may provide a longer-horizon clinical reference point for personalization of the system. In some embodiments, the lab A1C anchor 154 is the same or similar to the lab A1C input 20 described with respect to FIG. 1 and the onboarding baseline step 92 described with respect to FIG. 4. The lab A1C anchor 154 may be used to initialize the personalized model, constrain projection ranges, refine calibration, validate drift over time, or otherwise ground the system in a known clinical benchmark. In some embodiments, an updated laboratory A1C value obtained at a later date may be used to refresh or recalibrate the personalization / data-fusion engine 162, thereby allowing the model to remain aligned with the user's evolving glycemic status.

The CGM input 156 may include data obtained from a continuous glucose monitoring system. In some embodiments, the CGM input 156 is obtained through direct pairing, cloud synchronization, manual import, batch import, or another communication pathway. The CGM input 156 may include instantaneous glucose values, trend information, time-series history, excursion information, summary statistics, or event markers. In certain implementations, the system may use the CGM input 156 to correlate breath-derived input 152 with contemporaneous or near-contemporaneous glucose behavior, to identify divergence between a breath-derived trend and a wearable glucose trend, or to provide additional calibration points for the personalization / data-fusion engine 162.

The finger-stick input 158 may include one or more capillary glucose values or other discrete confirmatory readings entered manually by the user or obtained from a connected meter. In some embodiments, the finger-stick input 158 is used less frequently than the breath-derived input 152 and may be employed primarily during onboarding, recalibration, anomaly investigation, or specific user-requested validation events. Because finger-stick readings may provide discrete but relatively trusted reference points, the finger-stick input 158 may be particularly useful for cross-checking unexpected breath-derived results, anchoring model updates, or confirming whether a flagged session should meaningfully influence the user's rolling history.

The API / external data input 160 may include additional glycemic, metabolic, behavioral, or contextual information obtained from external systems. In some embodiments, the API / external data input 160 includes laboratory portal data, clinician-entered data, health-record data, wellness application data, nutrition logs, medication schedules, exercise records, sleep data, or other external information communicated through one or more application programming interfaces or data-transfer mechanisms. In some implementations, the API / external data input 160 may also include values derived from third-party analytics platforms, remote coaching services, or interoperable health-data infrastructures. The system may use such information to enhance interpretation of breath-derived trends or to generate richer reports, while still maintaining breath-derived input 152 as a central recurring data source.

The personalization / data-fusion engine 162 may receive and process any one or more of the inputs 152, 154, 156, 158, and 160. In some embodiments, the personalization / data-fusion engine 162 is implemented within the mobile application 16, a cloud environment associated with the remote data store 18, The breath-sensing 14, or a hybrid combination thereof. The personalization / data-fusion engine 162 may employ one or more algorithms configured to align, weight, compare, normalize, reconcile, and learn from multiple input types having different timing characteristics, confidence levels, and clinical significance.

In some embodiments, the personalization / data-fusion engine 162 assigns different weights to different input types. For example, a recent laboratory-derived A1C value may be used as a high-confidence longer-horizon anchor, a finger-stick value may be treated as a relatively high-confidence point reference for a specific time, a CGM stream may be treated as a dense temporal reference source, and breath-derived input 152 may be treated as a frequent, non-invasive recurring signal that becomes more individualized as repeated use accumulates. The relative influence of each input may vary according to recency, quality, source reliability, internal consistency, user state, or other factors.

In some implementations, the personalization / data-fusion engine 162 performs temporal alignment among different data sources. For example, the engine may associate a breath session with a contemporaneous CGM value, a nearby finger-stick value, a recent lab value, or external contextual information such as meal timing or exercise timing. Temporal alignment may be exact, approximate, window-based, event-based, or otherwise adapted to the available data. This may permit the engine to identify correlations, discrepancies, or trend relationships that would not be apparent from any one source in isolation.

The personalization / data-fusion engine 162 may also perform confidence scoring and conflict resolution. If two or more inputs appear mutually consistent, the system may increase confidence in a projected trend or score. If the inputs diverge meaningfully, the engine may reduce confidence, trigger the anomaly cross-check 168, request additional confirmation, or limit the influence of one or more questionable data points. In some embodiments, the engine uses session-quality flags, device status, source reliability, or known user-specific patterns to determine whether a particular input should be trusted, down-weighted, or ignored.

In certain embodiments, the personalization / data-fusion engine 162 supports incremental model updating. For example, the engine may begin with a lab A1C anchor 154 and early breath-derived sessions, then later incorporate occasional finger-stick input 158 or CGM input 156 to improve user-specific mapping between breath-derived signals and glycemic interpretation. In some implementations, the model may be updated continuously, periodically, after threshold events, or after receipt of new high-confidence external information. Such updating may help the system remain individualized as the user's physiology, medication regimen, lifestyle, or device-use patterns change over time.

The projected A1C output 164 may be generated by the personalization / data-fusion engine 162 based on any suitable combination of the available inputs. In some embodiments, the projected A1C output 164 is derived primarily from breath-derived input 152 and the lab A1C anchor 154, with optional refinement from the CGM input 156, finger-stick input 158, or API / external data input 160. The projected A1C output 164 may represent a numerical estimate, a value range, a directional change indicator, a band, or another longer-term glycemic indicator derived from the personalized fused model. In preferred embodiments, the projected A1C output 164 is presented as a modeled or projected indicator rather than as a direct laboratory measurement.

The daily score / zone output 166 may likewise be generated from the personalization / data-fusion engine 162 and may incorporate fused information when such information is available. In some embodiments, the daily score / zone output 166 is driven primarily by breath-derived input 152 together with rolling history, but may be adjusted or qualified based on external confirmation or contradiction from the CGM input 156, finger-stick input 158, or API / external data input 160. Thus, the daily score / zone output 166 may remain simple and user-facing while still benefiting from more sophisticated back-end fusion logic.

The anomaly cross-check 168 may be used to detect and respond to inconsistencies among the fused inputs. In some embodiments, the anomaly cross-check 168 examines whether a breath-derived session departs materially from expected behavior given the user's rolling history, lab anchor, prior fused model, or available external data. If a conflict is detected, the system may flag the session, reduce confidence, request a repeat session, request a confirmatory finger-stick or CGM review, suppress a user-facing score until further data is obtained, or otherwise modify system behavior. The anomaly cross-check 168 may therefore act as both a safety feature and a model-quality feature.

In some embodiments, the anomaly cross-check 168 distinguishes among different forms of inconsistency. A first form may reflect poor sample quality or environmental interference. A second form may reflect a sudden physiological change that is real but unusual. A third form may reflect inconsistency between breath-derived input 152 and one or more external data sources. The system may respond differently to each such condition. For example, a poor-quality breath session may trigger a repeat-session request, while a persistent divergence between breath-derived input and external reference data may trigger recalibration or clinician-oriented reporting.

The report / export output 170 may provide a fused summary of the information processed by the personalization / data-fusion engine 162. In some embodiments, the report / export output 170 includes one or more of the projected A1C output 164, the daily score / zone output 166, anomaly information from the anomaly cross-check 168, relevant historical trends, user-entered notes, quality indicators, and selected external-input summaries. The report / export output 170 may be formatted for user review, coaching, clinician discussion, telehealth use, research use, or another authorized purpose. In certain implementations, the report may indicate which external data sources were used, the relative recency of those sources, and whether the current outputs are based primarily on breath-derived information or on a broader fused dataset.

The data-fusion architecture may also operate gracefully when one or more external inputs are absent. For example, after onboarding, the system may continue to generate projected A1C-related outputs and daily progress indicators using breath-derived input 152 and prior personalization history even when no CGM input 156, finger-stick input 158, or API / external data input 160 is available. Conversely, when additional data sources are present, the system may opportunistically use them to improve calibration, detect drift, support confirmatory logic, and increase user or clinician confidence. The architecture is therefore flexible rather than dependent on any single supplemental source.

In some embodiments, the data-fusion architecture supports different user populations and deployment modes. A first user may rely almost entirely on breath-derived input 152 plus an occasional updated lab A1C anchor 154. A second user may additionally synchronize CGM data periodically. A third user may use finger-stick confirmation only during selected events. A clinician-supervised embodiment may incorporate richer API / external data input 160 and structured reporting. The same underlying personalization / data-fusion engine 162 may therefore support consumer, wellness, coaching, screening, and clinical-support use cases with different degrees of integration.

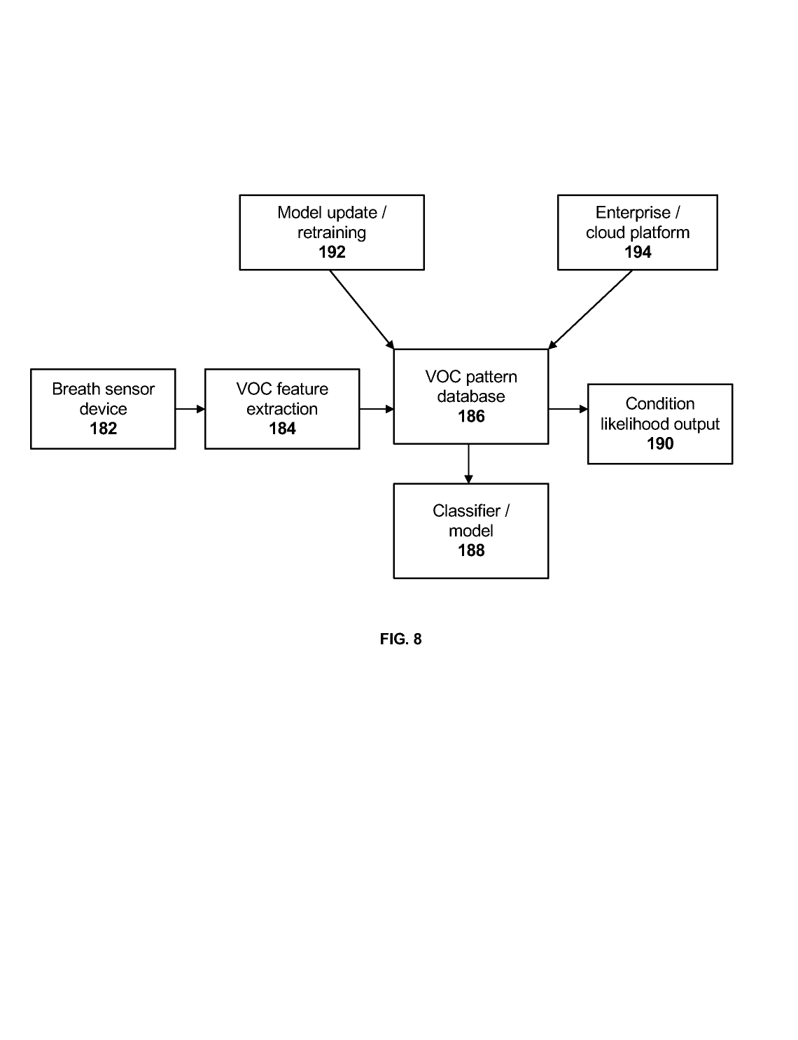

The data-fusion architecture shown in FIG. 7 thus provides a mechanism for integrating frequent, non-invasive breath-derived monitoring with one or more additional glycemic or contextual data sources in a manner that improves personalization without requiring such external sources for every session. By combining optional anchoring, cross-checking, recalibration, anomaly handling, and report generation, the architecture may enhance the practical utility of the disclosed monitoring platform. A broader VOC analytics architecture retained for additional support and possible continuation practice is described next with respect to FIG. 8.

### Broader VOC Analytics Architecture and Condition-Screening Embodiments

Referring to FIG. 8, a broader VOC analytics architecture is shown. In general, the VOC analytics architecture illustrates that the sensing, modeling, and reporting concepts disclosed herein may also be employed in applications extending beyond the personalized glycemic-monitoring embodiments described with respect to FIGS. 1-7. In the illustrated embodiment, the architecture includes a breath sensor device 182, a VOC feature extraction stage 184, a VOC pattern database 186, a classifier / model 188, a condition likelihood output 190, a model update / retraining stage 192, and an enterprise / cloud platform 194. The arrangement shown in FIG. 8 is illustrative rather than limiting, and the illustrated components may be implemented in whole or in part on-device, in a paired mobile application, in remote computing infrastructure, or in a distributed combination thereof.

The breath sensor device 182 may be the same as, similar to, derived from, or interoperable with The breath-sensing 14 described above. In some embodiments, the breath sensor device 182 includes one or more VOC sensors configured to respond to compounds present in exhaled breath, optionally together with one or more humidity sensors, temperature sensors, flow-related sensors, pressure sensors, or other environmental or quality-related sensors. In other embodiments, the breath sensor device 182 may be implemented in a different form factor, including a handheld instrument, tabletop device, kiosk device, clinical screening station, integrated vehicle or bedside unit, or other portable or fixed-use arrangement. The broader VOC analytics architecture is therefore not limited to a pendant configuration.

In operation, the breath sensor device 182 may acquire one or more breath samples from a user and generate sensor data corresponding to the sample or samples. In some embodiments, the device may also collect contextual information such as breath-session timing, sample duration, environmental conditions, device state, or user-entered notes. As in the glycemic-monitoring embodiments, the system may perform sample-quality evaluation, session rejection, normalization, drift handling, or confidence scoring before using the sensed information for further analysis. Thus, FIG. 8 preserves the broader principle that breath-derived data may be conditioned and qualified prior to downstream interpretation.

The VOC feature extraction stage 184 may receive raw or partially processed sensor data from the breath sensor device 182 and generate a feature representation suitable for comparison, classification, inference, or model updating. In some embodiments, the VOC feature extraction stage 184 includes signal filtering, baseline correction, temporal-response characterization, peak extraction, area-under-curve determination, ratio analysis, latent-feature generation, normalization against environmental variables, dimensionality reduction, signature generation, or another feature-engineering process. The resulting feature representation may be a scalar, a vector, a signature profile, a ranked set of sensed responses, a latent embedding, or another analytical construct that represents the sensed breath information in a form usable by the downstream model architecture.

In some embodiments, the VOC feature extraction stage 184 may additionally incorporate metadata or contextual variables. For example, the system may associate a feature representation with user demographics, time-of-day information, fasting status, medication timing, device identifier, session-quality information, confirmatory test information, or other annotations. Such metadata may improve later interpretation and may be especially useful in embodiments employing large comparative datasets, retraining workflows, or enterprise-level deployment. In other embodiments, the feature extraction stage 184 may be intentionally lightweight and may operate substantially on sensed VOC information alone.

The VOC pattern database 186 may store reference information against which newly derived VOC features can be compared. In some embodiments, the VOC pattern database 186 contains previously acquired breath-derived feature sets associated with one or more known states, conditions, classifications, outcomes, or labels. The database may include user-specific data, population-level data, cohort-specific data, device-specific calibration data, disease-state annotations, wellness-state annotations, laboratory-correlated records, clinician-reviewed records, or combinations thereof. In some implementations, the VOC pattern database 186 may be organized to support nearest-neighbor comparison, rules-based retrieval, similarity scoring, clustering, supervised learning, semi-supervised learning, or unsupervised pattern discovery.

The VOC pattern database 186 may be updated over time as new sessions and new labeled records become available. In some embodiments, the database may store confirmed examples associated with particular physiological or clinical states, while also storing unlabeled or weakly labeled examples for later use in retraining or exploratory analysis. The database may reside locally, remotely, or in a hybrid arrangement. In certain implementations, different portions of the database may be separated by device type, geography, user population, condition class, or regulatory environment.

The classifier / model 188 may receive the feature representation from the VOC feature extraction stage 184 and may compare the representation against one or more models, rules, thresholds, or comparative datasets associated with the VOC pattern database 186. In some embodiments, the classifier / model 188 is configured to identify whether the newly acquired breath session appears consistent with one or more reference patterns associated with a known condition, state, trend, or classification. The classifier / model 188 may take many forms, including a regression model, decision tree, ensemble method, support vector machine, Bayesian model, neural network, probabilistic classifier, rules-based engine, similarity model, ranking model, hybrid model, or another computational framework.

In some embodiments, the classifier / model 188 may generate more than one analytical output from a single breath session. For example, the model may generate one or more condition scores, ranked likelihoods, confidence measures, risk tiers, anomaly indicators, similarity measures, or recommended follow-up actions. In other embodiments, the classifier / model 188 may first determine whether a session is sufficiently reliable for interpretation before producing any downstream classification output. Thus, the classifier / model 188 may incorporate both inference logic and quality-aware gating logic.

The condition likelihood output 190 may represent one or more user-facing or clinician-facing results derived from the classifier / model 188. In some embodiments, the condition likelihood output 190 includes a likelihood score, category, rank ordering, confidence band, binary or multi-class decision, anomaly flag, trend label, or recommendation for confirmatory testing. The condition likelihood output 190 may be directed to a wide variety of use contexts. In some implementations, the output concerns glycemic status or metabolic trajectory. In other implementations, the output may relate to a broader metabolic condition, inflammatory state, respiratory state, infectious state, wellness status, screening outcome, or another VOC-associated classification.

In certain embodiments, the condition likelihood output 190 is intentionally simplified for a consumer-facing interface, such as through the use of zones, categories, or ranked outputs. In other embodiments, the condition likelihood output 190 may be more technical and may include confidence scores, similarity metrics, or feature-level explanations suitable for clinician review, research use, or enterprise analysis. The manner in which the output is rendered may therefore depend on the intended audience, use case, regulatory posture, and degree of model explainability available in the particular embodiment.

The model update / retraining stage 192 may allow the architecture to improve over time as new labeled or partially labeled information becomes available. In some embodiments, the model update / retraining stage 192 receives confirmed outcomes, external laboratory results, clinician annotations, confirmatory test results, user follow-up information, or later-obtained state labels associated with prior breath sessions. Such information may then be used to refine thresholds, retrain machine-learning models, update similarity metrics, rebalance cohorts, adjust feature weights, or otherwise improve performance. The model update / retraining stage 192 may operate continuously, periodically, upon accumulation of a threshold amount of new information, or in response to model-drift detection.

In some embodiments, retraining may occur using user-specific data, population-level data, or both. For example, a first level of updating may personalize a model to a particular user or subgroup, while a second level of updating may improve a broader population-level classifier. In certain implementations, user-specific adaptation and population-level model improvement may occur separately so that a consumer-facing personalized model is not necessarily changed every time a broader enterprise model is updated. This separation may be useful where the system supports both individualized monitoring and cross-user comparative analysis.

The enterprise / cloud platform 194 may support storage, analytics, synchronization, fleet management, reporting, clinician access, application integration, retraining workflows, and related back-end operations. In some embodiments, the enterprise / cloud platform 194 stores VOC feature records, user profiles, session metadata, model versions, quality flags, confirmatory results, report histories, and sharing permissions. The enterprise / cloud platform 194 may also provide interfaces to laboratories, healthcare providers, health-information systems, wellness platforms, remote coaching platforms, or research systems. In certain embodiments, the enterprise / cloud platform 194 may enforce privacy controls, access restrictions, audit logs, encryption, or other security and governance measures.

The enterprise / cloud platform 194 may further support cohort analysis or cross-user learning. For example, anonymized or permissioned records from multiple users may be used to refine reference patterns in the VOC pattern database 186, improve the classifier / model 188, or identify previously unrecognized VOC signatures associated with particular states or outcomes. In some implementations, the enterprise / cloud platform 194 may manage multiple deployment modes, such as a consumer mode, a clinician-supervised mode, a research mode, and an enterprise wellness mode, each of which may use different subsets of the same underlying architecture.

In some embodiments, the broader VOC analytics architecture may be used as a supplement to the glycemic-monitoring embodiments described previously. For example, the same breath sensor device may support both a personalized glycemic workflow and a more general VOC pattern-recognition workflow, with different outputs, models, or software modules enabled for different products or services. In other embodiments, the broader VOC analytics architecture may be practiced independently of any glycemic-monitoring function. Thus, FIG. 8 is retained not merely as background material, but as an embodiment showing that the disclosed sensing and modeling framework may support broader VOC analytics, screening, and classification applications.

The architecture may also support confirmatory and iterative workflows. For example, if the classifier / model 188 produces a condition likelihood output 190 having low confidence or identifying a potentially significant condition, the system may prompt for additional breath samples, external confirmatory testing, clinician review, or later follow-up. Any such subsequent information may then be fed back into the model update / retraining stage 192 and, where appropriate, into the VOC pattern database 186. In this way, the system may function not only as a single-pass inference engine but also as a learning and refinement platform.

In certain embodiments, the broader VOC analytics architecture may employ multiple classes of outputs simultaneously. A first output may be a personalized consumer-facing score or zone. A second output may be a more formal condition likelihood or ranking. A third output may be an internal quality or anomaly score used for gating or validation. These outputs may be generated from overlapping or distinct models and may be directed to different recipients. Accordingly, the system is not limited to a single result type.

The broader VOC analytics architecture shown in FIG. 8 therefore preserves a generalizable framework in which breath-derived VOC information is transformed into analytical features, compared against stored or learned patterns, and used to generate condition-related outputs that may be refined over time through retraining and enterprise-scale data handling. This broader architecture may coexist with, support, or extend beyond the personalized glycemic-monitoring embodiments described elsewhere in the present application. Additional embodiments, variations, and implementation options are described next.

### Preferred hardware implementations

In a preferred implementation, the breath-sensing device includes at least one gas sensor configured to generate a VOC-responsive signal from a user’s breath sample, and the gas sensor may be a semiconductor metal-oxide gas sensor or another gas-sensing element suitable for detecting one or more breath-borne compounds associated with glycemic status. In some embodiments, the device further includes a separate environmental sensor configured to detect both humidity and temperature, such that humidity and temperature information may be collected during a breath session and used in sample-quality evaluation, normalization, drift compensation, or confidence scoring. Although a preferred implementation may employ a particular combination of gas sensor and environmental sensor, other embodiments may use additional sensors, integrated sensors, or different sensor technologies while still performing the functions described herein.

In some embodiments, the printed circuit board carries control and communication electronics including a microcontroller and a wireless communication interface. In a preferred implementation, the communication interface supports short-range wireless communication with a paired mobile device, such as by Bluetooth Low Energy or another low-power wireless protocol. The control electronics may further include memory, analog front-end circuitry, signal conditioning circuitry, amplifier circuitry, digital-to-analog circuitry, analog-to-digital circuitry, clock circuitry, and related components used to acquire, condition, store, and communicate breath-session data. The particular component selection may vary across implementations and is not limited to any specific vendor, package, or part number.

In some embodiments, the device further includes a rechargeable internal power source, such as a single-cell rechargeable battery, together with associated charging, battery-management, protection, and voltage-regulation circuitry. Such circuitry may include one or more charging controllers, over-voltage protection components, over-current protection components, resettable protection devices, buck or linear regulators, switching devices, filtering components, and related power-management structures. The resulting power architecture may permit the device to operate as a compact portable product suitable for repeated daily use while maintaining stable operation of the sensing, control, and communication circuitry.

In some embodiments, the data / power port 82 is configured as a wired charging and/or data interface. In a preferred implementation, the data / power port 82 is a USB Type-C interface, although other wired connector types, charging pads, pogo-pin interfaces, docking contacts, or proprietary interfaces may also be used. The wired interface may support one or more of battery charging, wired data transfer, diagnostics, firmware update operations, factory programming, servicing, or accessory communication. In some embodiments, the device may communicate primarily wirelessly during ordinary use while retaining the wired interface for charging, service, or accessory-related functions.

In some embodiments, the device further includes one or more user-feedback components. For example, the status indicator 54 may include one or more light-emitting devices, such as a single-color or multi-color LED, and the device may further include an audible output component such as a buzzer and/or a tactile feedback component such as a vibration motor. These feedback components may be used to indicate device readiness, active sampling, session completion, low-confidence sessions, pairing state, charging state, low-battery state, anomaly conditions, confirmatory prompts, or other operating conditions. In some embodiments, such outputs may supplement, or in certain situations replace, information shown on a paired mobile application.

### Additional Embodiments, Implementation Options, and Variations

The embodiments described above are illustrative and may be implemented in a wide variety of alternative forms without departing from the scope of the present disclosure. Although many examples herein describe a pendant-style Breath-sensing 14 used in conjunction with a mobile application 16 and, in some embodiments, a remote data store 18 or personalization / data-fusion engine 162, the disclosed subject matter is not limited to that particular product architecture. The same or similar sensing, personalization, longitudinal analysis, and reporting concepts may be embodied in other wearable, portable, stationary, or distributed systems.

For example, the breath-sensing hardware may be implemented as a pendant, lanyard device, keychain device, handheld unit, desktop unit, bedside unit, kiosk unit, vehicle-mounted unit, dockable unit, clip-on device, or another consumer or clinical form factor. In some embodiments, the device is configured primarily for personal daily use. In other embodiments, the device may be configured for supervised use in a clinic, wellness center, pharmacy, workplace, research site, or other testing environment. The device enclosure, materials, inlet geometry, attachment structures, and external interface may therefore vary substantially across implementations.

Likewise, the internal sensing architecture may vary. In some embodiments, the device includes a single VOC sensor and one or more additional environmental sensors. In other embodiments, the device includes multiple VOC sensors, multiple sensor types, a sensor array, or multiple partially redundant sensing channels. Additional sensors may include humidity sensors, temperature sensors, flow-related sensors, pressure sensors, proximity sensors, motion sensors, or other components useful for evaluating sample quality, context, or environmental conditions. The particular selection of sensing components may be chosen according to cost, manufacturability, stability, intended use, desired performance, power budget, or regulatory posture.

The breath-collection arrangement may also vary. In some embodiments, the user exhales directly toward an exposed breath inlet region 46. In other embodiments, the device may include a mouthpiece, replaceable mouth-contact component, nozzle, tube, channel, shielded inlet, directional inlet, removable breath guide, or another sample-guidance structure. Some embodiments may use a single exhalation per session, while other embodiments may use multiple exhalations, repeated trials, preconditioning exhalations, or adaptive session structures in which the system requests additional breath samples based on sample-quality results. The duration, timing, spacing, and acceptance criteria for breath collection may be fixed, semi-fixed, user-guided, or dynamically adjusted.

In some embodiments, the device may include one or more filters, membranes, barriers, purge paths, vent structures, or passive or active flow-management components. These may be used to reduce particulate contamination, reduce liquid exposure, encourage gas-phase delivery to a sensing region, evacuate residual gas, or improve repeatability from session to session. In other embodiments, the internal stack may be simpler or more integrated than the example arrangement shown in FIG. 3. Thus, the exploded view described above should be understood as illustrative rather than restrictive.

Power and communication arrangements may also vary. In some embodiments, the device includes a rechargeable power source and wireless communication circuitry. In other embodiments, the device may support wired communication, docking-based communication, inductive charging, contact-based charging, battery replacement, or low-power intermittent synchronization. The system may operate in a continuously connected mode, an intermittently synchronized mode, or a largely offline mode in which data is stored locally and uploaded later. Similarly, firmware updates, diagnostics, and calibration routines may be performed wirelessly, through a dock, through a wired port, or through other service interfaces.

The processing architecture may likewise be distributed in different ways. In some embodiments, most breath interpretation logic is performed on the device itself. In other embodiments, the device primarily gathers data while the mobile application 16 performs normalization, personalization, scoring, and display functions. In still other embodiments, some or all model execution may occur in cloud-based computing resources associated with the remote data store 18. Accordingly, functions described herein as occurring in a particular system component may instead be performed in another component or divided among multiple components. The claimed subject matter is not limited to any one allocation of computing responsibility.

The personalization approach may also vary across embodiments. In some implementations, personalization begins with a user-entered or imported lab A1C value and is refined using repeated breath sessions. In other implementations, personalization may additionally or alternatively use one or more laboratory glucose values, finger-stick values, continuous glucose monitor data, clinician-provided inputs, prior session history, demographic information, medication information, behavioral context, or user-specific calibration routines. In some embodiments, the personalization engine is designed to begin with limited information and improve as more sessions accumulate. In other embodiments, the system may require a threshold amount of baseline information before certain user-facing outputs are enabled.

The manner in which a lab A1C value is used may also vary. In some embodiments, the lab A1C value serves as an initialization anchor for a user-specific model. In some embodiments, a later laboratory A1C value may be used to recalibrate or validate the personalized model after a period of use. In other embodiments, multiple laboratory values over time may be used to refine trend interpretation or detect drift between the projected A1C output and a clinically obtained benchmark. The disclosure therefore encompasses both one-time anchoring and repeated recalibration approaches.

The system may use a wide variety of analytical methods. In some embodiments, the personalization / data-fusion engine 162 or classifier / model 188 employs rules-based logic, thresholding, scoring logic, signal transforms, or regression-based relationships. In other embodiments, the engine or model may employ machine-learning techniques, neural-network techniques, probabilistic modeling, ranking methods, similarity analysis, adaptive filtering, or hybrid techniques that combine deterministic logic with learned relationships. The disclosed subject matter is not limited to any particular analytical paradigm. Moreover, different models may be used for different functions, such as sample-quality evaluation, session normalization, daily scoring, projected A1C-related inference, anomaly detection, or broader VOC pattern recognition.

In some embodiments, a first model is user-specific and adaptive, while a second model is population-based, cohort-based, or device-calibration-based. These different model layers may cooperate. For example, a user-specific model may provide day-to-day scoring, while a broader model may provide starting parameters, anomaly benchmarks, or classifier outputs for broader VOC analytics. In some embodiments, the system may update the user-specific model more frequently than the broader model. In other embodiments, the broader model may be updated centrally while the user-specific model remains relatively stable between recalibration events.

The system may also use different weighting or confidence approaches. A recent high-quality breath session may be given greater influence than an older or lower-quality session. A laboratory A1C value may be given more weight than a user-entered unverified value. A confirmatory finger-stick reading may be treated as more influential for a specific time point than a routine breath session. A CGM stream may be used as a dense temporal reference in some embodiments but may be entirely absent in others. Any suitable weighting, recency adjustment, confidence scoring, or source-reliability logic may be employed.

The outputs presented to the user may likewise take many forms. In some embodiments, the system presents a numerical score. In other embodiments, the system presents a zone, band, color-coded range, ranked category, trend arrow, icon, message, or another simplified progress-oriented output. The projected A1C-related output may be displayed as a number, a range, a trajectory, a change indicator, a confidence-qualified estimate, or another presentation suitable for user understanding. Some embodiments may emphasize motivation and simplicity, while other embodiments may provide more technical detail for clinician review, coaching, or structured reporting.

The system may also support different alerting behaviors. In some embodiments, the system notifies the user when a session is complete, when a repeat session is recommended, when a confirmatory reading is advisable, when a trend is improving, when a trend is worsening, or when a report is available. Alerts may be delivered through visual, audible, haptic, or remote-notification channels. In some embodiments, the system may intentionally suppress alerts when confidence is insufficient, or may present alerts only after repeated confirming conditions are detected. The exact thresholds and messaging policies may vary.

The notes, reporting, and export features may also be implemented in many ways. In some embodiments, the system permits free-text note entry. In other embodiments, the system uses structured tags, preconfigured menus, dictated input, or automatically suggested contextual labels. Reports may be generated on demand, on a schedule, in response to an anomaly, after a clinician request, or at the end of a selected time interval. Reports may be formatted for user comprehension, clinician review, coaching, research, enterprise wellness, remote consultation, or insurance or reimbursement support where applicable. Export may occur through portable files, secure messages, application interfaces, clinician portals, or other authorized pathways.

Privacy, authorization, and data-governance features may also vary. In some embodiments, user data is encrypted at rest and in transit. In some embodiments, export and sharing require user authorization and may be limited to approved recipients. Certain embodiments may support de-identification, anonymization, or pseudonymization for model training, research, or cohort analysis. Other embodiments may maintain user-specific audit logs, permission histories, or data-retention controls. Although such features are not shown in detail in every figure, they may be incorporated into any embodiment described herein.

The disclosed system may be deployed in different operating contexts. In one embodiment, the system is used primarily as a consumer-facing wellness and behavioral-motivation platform. In another embodiment, it is used as a home-monitoring adjunct in conjunction with clinician care. In another embodiment, it is used as a screening or triage aid that helps identify when confirmatory testing may be appropriate. In yet another embodiment, the architecture may be adapted for broader VOC-based monitoring or classification workflows beyond glucose-related interpretation. Accordingly, the system may support consumer, research, enterprise, screening, clinical-support, and mixed-use deployments.

In some embodiments, the breath-sensing device may be carried or mounted in different ways depending on the intended use environment. For example, the device may be configured for pendant use with a lanyard, for clipped use with a clip or clothing attachment, for pocket or bag carriage, or for temporary placement in a holder, dock, or stand. In certain embodiments, the device may be used with a charging cradle, charger stand, docking accessory, bedside stand, desktop stand, or other support structure that positions the device for charging, storage, transport, or repeated access throughout the day. Such accessories may be passive supports or may include charging or communication features.

In some embodiments, a clip-based carrying configuration may be provided in addition to, or instead of, a lanyard-based carrying configuration. A clip, clasp, magnetic attachment, carabiner-style attachment, adhesive mount, or other securement structure may permit the user to couple the device to clothing, a bag, a keychain, a vehicle surface, exercise equipment, or another convenient location. The carrying or mounting arrangement is therefore not limited to any one wearable geometry.

In some embodiments, the device may be configured to cooperate with a charging dock or charger stand through the data / power port 82 or through another electrical interface. The charging dock or charger stand may be configured to recharge the internal power source, provide a convenient storage position, support wired diagnostics or firmware servicing, or maintain the device in a known orientation when not actively in use. In certain embodiments, the dock or stand may be used in home, bedside, desktop, vehicle, or clinic environments.

In some embodiments, alert outputs may be generated using one or more of visual, audible, and tactile notification channels. For example, the device may illuminate one or more LEDs, emit a buzzer tone, produce vibration, or generate a combination of such outputs in response to readiness states, active sampling states, acceptable sample completion, low-confidence sample conditions, anomaly conditions, threshold events, confirmatory test prompts, synchronization events, charging events, or low-power events. Different alert modalities may be selected according to user preference, accessibility considerations, environment, or power-management criteria.

In some embodiments, the system may permit a user to configure which local alert mechanisms are enabled. For example, a user may choose visual-only alerts in one environment, haptic alerts in another environment, or combined visual, audible, and tactile alerts when more immediate notice is desired. Local device alerts may also be coordinated with alerts generated by a paired mobile application so that the user receives a consistent experience across the device and the companion software environment.

In some embodiments, a total sample count, total breath processing count, total power on/off, or total active time the breath sensor is in use may be logged. Such counts may be used to monitor the longevity of certain critical components, or to prevent use of the device if certain longevity periods are exceeded. For instance, the sensor may be certified for 500 uses or a set amount of up time, after which it must be replaced. This can be accounted for by the system, and use of the device can be halted until the part is changed or the counter is reset.

In some embodiments, the disclosed methods may be implemented, at least in part, as software instructions stored on one or more non-transitory computer-readable media and executed by one or more processors. Such processors may be located in The breath-sensing 14, a mobile device running the mobile application 16, a server associated with the remote data store 18, or another computing environment. The software instructions may cause one or more processors to perform operations such as acquiring breath-session data, evaluating sample quality, normalizing sensor information, deriving glucose-correlated breath metrics, initializing or updating personalized models, generating projected A1C-related outputs, generating zone or progress outputs, performing anomaly checks, fusing external data, and generating reports or exportable summaries.

In some embodiments, the disclosed subject matter may be practiced as a system, as a method, as one or more devices, as a computer-readable medium storing executable instructions, or as a combination of such statutory classes. Features described in connection with one embodiment may be combined with features of another embodiment unless expressly incompatible. Likewise, functions illustrated as separate blocks may be combined in a single module, and functions illustrated in a single block may be distributed across multiple modules.

It should also be understood that terms such as "may," "can," "configured to," "adapted to," and similar language are used to describe example capabilities and are not intended to imply that every embodiment must include every described feature. Similarly, reference to a component in the singular may encompass one or more such components, and reference to multiple components may in some cases be satisfied by an integrated component performing the corresponding functions. Relative terms such as "front," "rear," "upper," "lower," "inward," and "outward" are used for convenience with reference to illustrated embodiments and are not intended to require any particular orientation except where expressly stated.

Unless the claims expressly require otherwise, no feature described in this description should be understood as essential in all embodiments. The scope of protection should instead be determined from the claims as ultimately presented, together with all permissible equivalents. The present disclosure therefore encompasses the specific embodiments described above, modifications thereof, combinations thereof, and other implementations that fall within the spirit and scope of the claimed subject matter.

What is claimed is:

1. A method for non-invasive glycemic monitoring of a user, the method comprising:
receiving a lab-derived A1C baseline for the user;
acquiring, with a breath-sensing device, a plurality of breath samples from the user over time;
detecting, via the breath-sensing device, volatile organic compound (VOC) data and at least one of humidity data or temperature data from the plurality of breath samples;
evaluating sample quality for at least a subset of the plurality of breath samples;
normalizing the VOC data for accepted breath samples using the at least one of humidity data or temperature data to derive a glucose-correlated breath metric for each accepted breath sample;
initializing a personalized model for the user using the lab-derived A1C baseline and at least one glucose-correlated breath metric;
storing the lab-derived A1C baseline and the accepted breath samples in a rolling history;
updating the personalized model using additional accepted breath samples in the rolling history; and
using the personalized model and the rolling history to generate, for display via a user interface, (i) a projected A1C trend and (ii) a daily progress score or zone output representative of a glycemic trajectory of the user.

2. The method of claim 1, wherein receiving the lab-derived A1C baseline comprises receiving the lab-derived A1C baseline by manual entry, clinician entry, laboratory-portal import, electronic health record import, or application programming interface import.

3. The method of claim 1, wherein the plurality of breath samples are acquired during at least one session, and wherein multiple breath samples from the at least one session are selected or combined to derive a representative glucose-correlated breath metric for the session.

4. The method of claim 1, wherein evaluating sample quality comprises rejecting or down-weighting a breath sample based on insufficient exhalation, excessive moisture, unstable sensor response, or environmental conditions falling outside an acceptable range.

5. The method of claim 1, wherein updating the personalized model using the rolling history comprises weighting more recent accepted breath samples more heavily than older accepted breath samples.

6. The method of claim 1, further comprising performing a threshold or anomaly check on at least one of the accepted breath samples, the rolling history, the projected A1C trend, or the daily progress score or zone output.

7. The method of claim 6, further comprising, responsive to the threshold or anomaly check, issuing a confirmatory test prompt requesting at least one of a repeat breath sample, a finger-stick reading, a continuous glucose monitor reading, or a laboratory test.

8. The method of claim 1, further comprising generating a report or export output that includes at least the projected A1C trend, the daily progress score or zone output, and a history of prior accepted breath samples.

9. A non-invasive glycemic monitoring system, comprising:
a breath-sensing device comprising a housing, a breath inlet region, at least one volatile organic compound (VOC) sensor, at least one humidity sensor, at least one temperature sensor, a processor, and a wireless communication interface; and
at least one computing device in communication with the breath-sensing device and storing instructions that, when executed, cause the at least one computing device to:
receive a lab-derived A1C baseline for a user;
receive breath-session data from the breath-sensing device for a plurality of breath sessions acquired from the user over time;
evaluate sample quality and normalize the breath-session data using humidity data and temperature data to derive glucose-correlated breath metrics for accepted breath sessions;
initialize and update a personalized model for the user using the lab-derived A1C baseline and the glucose-correlated breath metrics;
store a rolling history of the accepted breath sessions; and generate, for presentation in a user interface, a projected A1C trend and a daily progress score or zone output.

10. The system of claim 9, wherein the breath-sensing device is a wearable pendant device comprising an attachment loop and a status indicator.

11. The system of claim 9, wherein the breath-sensing device further comprises a front cover, a dust filter, and a hydrophobic barrier arranged such that exhaled breath passes through the breath inlet region toward the at least one VOC sensor.

12. The system of claim 9, further comprising a remote data store configured to maintain the lab-derived A1C baseline, the rolling history, and one or more model parameters associated with the personalized model.

13. The system of claim 9, wherein the user interface comprises a dashboard view, a projected A1C tile, a history or trend view, a notes control, and a report or export view.

14. The system of claim 9, wherein the instructions further cause the at least one computing device to perform a threshold or anomaly cross-check and, responsive thereto, issue a confirmatory test prompt.

15. The system of claim 9, wherein the instructions further cause the at least one computing device to receive at least one external glucose input selected from a continuous glucose monitor input, a finger-stick input, or an external application programming interface input, and to use the at least one external glucose input in a personalization or data-fusion process.

16. A non-transitory computer-readable medium storing instructions that, when executed by one or more processors, cause the one or more processors to:
receive breath-derived input from repeated breath sessions of a user;
receive a lab-derived A1C anchor for the user;
receive at least one external glucose input selected from a continuous glucose monitor input, a finger-stick input, and an external application programming interface input;
use a personalization or data-fusion engine to combine the breath-derived input, the lab-derived A1C anchor, and the at least one external glucose input to generate a projected A1C output and a daily score or zone output for the user;
perform an anomaly cross-check on at least one of the breath-derived input, the projected A1C output, or the daily score or zone output; and
generate a report or export output based on at least the projected A1C output and the daily score or zone output.

17. The non-transitory computer-readable medium of claim 16, wherein the instructions further cause the one or more processors to temporally align at least one repeated breath session with at least one of the continuous glucose monitor input or the finger-stick input.

18. The non-transitory computer-readable medium of claim 16, wherein the personalization or data-fusion engine applies different weights to the breath-derived input, the lab-derived A1C anchor, and the at least one external glucose input based on recency, source reliability, or sample quality.

19. The non-transitory computer-readable medium of claim 16, wherein the instructions further cause the one or more processors to reduce confidence in, qualify, or suppress the daily score or zone output when the anomaly cross-check identifies an inconsistency among fused inputs.

20. The non-transitory computer-readable medium of claim 16, wherein the instructions further cause the one or more processors to update or retrain at least part of the personalization or data-fusion engine using confirmatory information obtained after the anomaly cross-check.
